(19) United States
(12) Patent Application Publication
(10) Pub. No.: US 2021/0380936 A1
(43) Pub. Date: Dec. 9, 2021

(54) An Implantable Construct, Methods of Manufacturing, and Uses Thereof

(71) Applicant: AGENCY FOR SCIENCE, TECHNOLOGY AND RESEARCH, Singapore (SG)

(72) Inventors: Youshan Melissa Lin, Singapore (SG); Steve Kah Weng Oh, Singapore (SG); Shaul Reuveny, Singapore (SG); William Richard Nicholas Birch, Singapore (SG); Jian Li, Singapore (SG); Asha Shekaran, Singapore (SG); Tin Lun Alan Lam, Singapore (SG)

(21) Appl. No.: 17/288,140

(22) PCT Filed: Oct. 23, 2019
(86) PCT No.: PCT/SG2019/050524

§ 371 Date: Apr. 23, 2021

(30) Foreign Application Priority Data
Oct. 23, 2018 (SG) 10201809364P

(51) Int. Cl. C12N 5/00; C12N 5/077; A61L 27/36

(52) U.S. Cl. CPC C12N 5/0075; C12N 5/0655; A61L 27/3654; A61L 2430/06; C12N 2531/00; C12N 2533/30; C12N 2506/1353; A61L 27/3612

(57) ABSTRACT

The present invention refers to a method of manufacturing an implantable construct comprising chondrogenically differentiated cells and one or more polycaprolactone (PCL) microcarriers, an implantable construct produced using said method, and uses of the implantable construct. The present invention also refers to a method of manufacturing an implantable construct comprising mesenchymal stromal cells and one or more polycaprolactone (PCL) microcarriers, an implantable construct produced using said method, and uses of the implantable construct. The present invention further refers to a method of treating a disease or disorder associated with cartilage and/or bone defect, the method comprises administering one or more cell-free polycaprolactone (PCL) microcarriers in a patient suffering from the disease or disorder.

### CROSS-REFERENCE TO RELATED APPLICATIONS

This application claims the benefit of priority of the Singapore application No. 10201809364P, filed on 23 Oct. 2018, the contents of it being hereby incorporated by reference in its entirety for all purposes.

### FIELD OF THE INVENTION

The present invention relates to the fields of cell biology, molecular biology and biotechnology. More particularly, the present invention relates to the culturing of stem cells on microcarriers.

### BACKGROUND OF THE INVENTION

Cartilage diseases and bone diseases in the broadest sense describe a group of diseases that are characterized by degeneration of or metabolic abnormalities in the connective tissue manifested by pain, stiffness and limitation of motion of the affected body parts. The origin of these disorders can be pathological or even a result of trauma or injury.

Because mature chondrocytes and osteocytes have little potential for replication, mature cartilage and bones have only a limited ability to restore themselves. For this reason, transplantation of cartilage tissue, isolated chondrocytes, bone tissue or isolated osteocytes into damaged cartilage and bones have been used therapeutically.

In recent years, stem cells such as mesenchymal stromal cells (MSCs) have shown promise for multiple therapeutic applications. However, translating these therapies into the clinical setting is hindered by challenges in scalable and reproducible manufacturing of MSCs at volumes that can meet clinical demand, as well as the lack of integrative bioprocesses for the expansion and delivery of MSCs, given that clinical applications require sizeable MSCs doses.

Classical methods of expanding MSCs for industrial applications in 2D monolayer flasks offer modest cell productivity. They are less suited to culture monitoring and require laborious, time-consuming handling, which makes such methods unsuitable for meeting the requirements of clinical applications. Another approach involves expanding MSCs on cell culture supports and subsequently harvesting the expanded MSCs from the cell culture supports using enzymatic digestion and/or mechanical dissociation. However, such harvesting procedures have detrimental impacts on the MSCs, and the MSCs obtained as such generally require a recovery time from the harvesting procedure before resuming their full functional potential. Thus, it is an object of the present invention to provide alternative methods to produce implantable constructs, preferably those containing stem cells such as MSCs, for use in the therapeutic treatment of cartilage diseases and bone diseases.

### SUMMARY OF THE INVENTION

In one aspect of the present invention, there is provided a method of manufacturing an implantable construct comprising chondrogenically differentiated cells and one or more polycaprolactone (PCL) microcarriers, the method comprises: a) culturing mesenchymal stromal cells with one or more PCL microcarriers in a suspension culture in a mesenchymal stromal cells growth medium to allow the mesenchymal stromal cells to attach to the PCL microcarriers to form one or more mesenchymal stromal cells-PCL microcarrier complexes, wherein the suspension culture is agitated; b) harvesting the one or more mesenchymal stromal cells-PCL microcarrier complexes from the suspension culture in a) while the suspension culture is agitated; c) culturing the one or more mesenchymal stromal cells-PCL microcarrier complexes from b) under agitation-free and centrifugation-free conditions in the mesenchymal stromal cells growth medium; d) culturing the one or more mesenchymal stromal cells-PCL microcarrier complexes from c) under agitation-free and centrifugation-free conditions in a chondrogenic differentiation medium to enact differentiation of the mesenchymal stromal cells into chondrogenically differentiated cells.

In another aspect, there is provided an implantable construct comprising chondrogenically differentiated cells and one or more PCL microcarriers, produced using the method as described above. In another aspect, there is provided an implantable construct comprising chondrogenically differentiated cells and one or more PCL microcarriers, wherein the number of chondrogenically differentiated cells per PCL microcarrier is about 10 to about 30.

In another aspect, there is provided a method of treating a disease or disorder associated with cartilage defect, the method comprises administering the implantable construct as described above in a patient suffering from the disease or disorder.

In another aspect, there is provided a method of promoting cartilage tissue regeneration in a patient in need thereof, the method comprises administering the implantable construct as described above in the patient.

### BRIEF DESCRIPTION OF THE DRAWINGS

The invention will be better understood with reference to the detailed description when considered in conjunction with the non-limiting examples and the accompanying drawings, in which:

FIG. 1 Evaluation of critical parameters required to achieve efficient chondrogenic differentiation of heMSC-LPCL microcarrier constructs. FIG. 1A shows brightfield images (scale bar, 100 μm) and FIG. 1B shows kinetics of heMSC growth on LPCL microcarriers in agitated spinner culture. Numbers in parentheses indicate the cell confluency (dotted line represents 100% cell confluency of 4.7×10⁴ cells/cm² as calculated from monolayer cultures). Arrows indicate the timepoints where cells-laden microcarriers taken from spinner culture were used to seed heMSC-LPCL constructs. The results show that seeding 5×10⁴ cells at 21% confluency per hMSC-LPCL construct resulted in most efficient cell growth.

FIG. 2 shows that seeding 50×10³ cells at 21% cell confluency per heMSC-LPCL construct (grey circle) resulted in efficient cell growth and chondrogenic differentiation by 21 days of differentiation. FIG. 2A shows DNA, FIG. 2B shows GAG, and FIG. 2C shows collagen II content per construct by day 21 of differentiation as well as respective fold increases from day 0 to day 21 of differentiation. The results show that seeding 5×10⁴ cells at 21% confluency per hMSC-LPCL construct resulted in most efficient cell growth (measured in terms of DNA content and fold increase), and most efficient chondrogenic differentiation (measured in terms of GAG and Collagen II content and fold increase).

FIG. 3 shows that construct compaction by applying centrifugation at seeding or continuous agitation throughout differentiation attenuate cell growth and reduce chondrogenic output. FIG. 3A shows DNA, FIG. 3B shows GAG, and FIG. 3C shows collagen II content per construct at day 21 of differentiation and relevant fold increases from day 0 to day 21 of differentiation.

FIG. 4 shows heMSC-LPCL constructs increased cellular proliferation and improved total chondrogenic output in terms of proteoglycan and Collagen II content, as compared to their equivalent cells-only counterparts. Kinetics of DNA (FIG. 4A), GAG (FIG. 4B) and Collagen II (FIG. 4C) production per construct were monitored during 28 days of differentiation. All p-values refer to statistical significance obtained by comparing heMSC-LPCL constructs over that of cells-only counterparts at indicated timepoints. P-values, n.s.=p>0.05, *p<0.05, **p<0.01, ***p<0.001 and ****p<0.0001.

FIG. 5 shows H&E (Haemotoxylin and Eosin) staining results that revealed best cartilage healing outcomes at 5 months post-transplantation with chondrogenically differentiated heMSC-LPCL constructs (black box). Percentages refer to proportion of joints with either poor (left column) or good (right column) healing outcomes. Scale bar, 1 mm.

FIG. 6 shows Safranin O staining results that revealed best cartilage healing outcomes at 5 months post-transplantation with chondrogenically differentiated heMSC-LPCL constructs (black box). Percentages refer to proportion of joints with either poor (left column) or good (right column) healing outcomes. Scale bar, 1 mm.

FIG. 7 shows Alcian Blue staining results that revealed best cartilage healing outcomes at 5 months post-transplantation with chondrogenically differentiated heMSC-LPCL constructs (black box). Percentages refer to proportion of joints with either poor (left column) or good (right column) healing outcomes. Scale bar, 1 mm.

FIG. 8 shows Masson's Trichrome staining results that revealed best cartilage healing outcomes at 5 months post-transplantation with chondrogenically differentiated heMSC-LPCL constructs (black box). Percentages refer to proportion of joints with either poor (left column) or good (right column) healing outcomes. Scale bar, 1 mm.

FIG. 9 shows Collagen II immunostaining results that revealed best cartilage healing outcomes at 5 months post-transplantation with chondrogenically differentiated heMSC-LPCL constructs (black box). Percentages refer to proportion of joints with either poor (left column) or good (right column) healing outcomes. Scale bar, 1 mm.

FIG. 10 shows growth kinetics and MSC surface marker expression of heMSCs expanded on LPCL in spinner flask cultures. FIG. 10A shows growth kinetics during 6-day expansion. heMSCs were cultured to 50% confluence at day 3 and 100% confluence at day 6. FIG. 10B shows expression of MSC markers CD34, CD45, CD73, CD90 and CD105 by heMSCs cultured on LPCL on day 3 (50% confluence) and day 6 (100% confluence). The results show that the highest cell density was reached on day 6, when cells achieved 100% confluence. The results also show that heMSCs harvested from 50% confluence LPCL and 100% confluence LPCL culture displayed high (80%-90%) levels of MSC makers CD73, CD90, and CD105, with low levels of CD34 and CD45.

FIG. 11 shows comparison of cytokine specific production rate of heMSCs from 50% heMSC-covered LPCL (mid log phase) and 100% cell-covered LPCL (stationary phase) in spinner flask cultures. ***p<0.001; ****p<0.0001. The results show that Subconfluent, mid logarithmic (50% confluency) and confluent, stationary (100% confluency) heMSC-covered LPCL exhibit different levels of cytokines production. Increasing cell density and the attainment of confluency, in the stationary phase, gives rise to a marked decreased in the specific production rate of cytokines.

FIG. 12 shows Micro-CT reconstructions (FIG. 12A) and quantification of bone volume (FIG. 12B) in excised implants at 16 weeks after calvarial defect implantation in mice. Five implant conditions were tested: (1) empty defects as a control (Empty control), (2) defect filled with cell-free LPCL (LPCL only), (3) defect filled with MSCs harvested from MNL cultures (MNL MSCs), (4) defect filled with 100% heMSCs-covered LPCL (100% MSCs LPCL), (5) defect filled with 50% heMSCs-covered LPCL (50% MSCs LPCL) and (6) Autograft (benchmark) defect. Data are mean with standard error (n=3-5). Statistical analysis was performed by analysis of variance and pairwise comparisons with post hoc Tukey correction in GraphPad. **p<0.01 and ***p<0.001. The results show that defect treated with cell-free LPCL yielded a low value of bone volume. The monolayer MSCs group gave rise to modest organized mineralized regions, with no significant differences in overall regrown bone volume, as compared with the untreated empty defect group. In contrast, the 100% MSCs LPCL group demonstrated significant mineralized tissue formation within the defect area. This is more than two-fold higher than the monolayer MSCs group. 50% MSCs LPCL group demonstrated dramatically better mineralized tissue formation within the defect area, when compared to the 100% MSCs LPCL group.

FIG. 13 shows H&E stains evaluation of excised implants at 16 weeks after calvarial defect implantation in mice. Five implant conditions were tested: (1) empty defects as a control (Empty control), (2) defect filled with cell-free LPCL (LPCL only), (3) defect filled with MSCs harvested from MNL cultures (MNL MSCs), (4) defect filled with 100% heMSCs-covered LPCL (100% MSCs LPCL), (5) defect filled with 50% heMSCs-covered LPCL (50% MSCs LPCL) and (6) autograft. Red circle (dotted) indicates putative capillary formation, while arrows indicate osteoclast bone remodeling. 100× magnification (Scale bar=100 μm). The results show that the untreated open defect remained unfilled. In contrast, both heMSCs-covered LPCL groups demonstrated more bone formation at the defect peripheries.

FIG. 14 shows Masson's trichrome staining of excised implants at 16 weeks after calvarial defect implantation in mice under 20× magnification (FIG. 14A) and 100× (FIG. 14B) magnification. Five implant conditions were tested: (1) empty defects as a control (Empty control), (2) defect filled with cell-free LPCL (LPCL only), (3) defect filled with MNL MSCs, (4) defect filled with 100% MSCs LPCL, (5) defect filled with 50% MSCs LPCL, and (6) autograft. Implants were paraffin embedded, sectioned to 5-um thickness and stained with Masson's Trichrome. The results show that tissue formation was different across the groups that introduced MSCs. In addition to the denser tissue formation, heMSCs-covered LPCL exhibited greater production of connective tissue. In addition, more connective tissue was observed in the 50% MSCs LPCL group than the 100% MSCs LPCL group.

### DEFINITIONS

The term “polycaprolactone” or the short form “PCL” as used herein refers to a biodegradable polyester, preferably has the molecular formula (C₆H₁₀O₂)ₙ. It has a low melting point of around 60° C. and a glass transition temperature of about −60° C. PCL has a density of 1.145 g/cm³ under standard conditions (i.e. 25° C. and 100 kPa). PCL is prepared by ring opening polymerization of ε-caprolactone using a catalyst such as stannous octoate. PCL is degraded by hydrolysis of its ester linkages in physiological conditions (such as in the human or animal body) and is therefore suitable for use as an implantable biomaterial. The term “LPCL” as used herein in describing the microcarrier refers to “light” PCL microcarrier, i.e. a PCL microcarrier with inner pores, resulting in a PCL microcarrier with lower overall density than a PCL microcarrier without inner pores. Since a PCL microcarrier without any inner pores has the same density as PCL under standard conditions, i.e. a density of 1.145 g/cm³, a LPCL microcarrier has a density of lower than 1.145 g/cm³. A LPCL microcarrier in general has a higher density than its surrounding fluid. For example, if the surrounding fluid of the LPCL microcarrier is water or is a cell culture medium having the same density as water, then the overall density of a LPCL microcarrier is higher than the density of water, e.g. higher than 1 g/cm³ under standard conditions.

The term “mesenchymal stromal cells” or the short form “MSCs” as used herein refers to multipotent stromal cells (i.e. connective tissue cells) that can differentiate into a variety of cell types, including, for example, osteoblasts (bone cells), chondrocytes (cartilage cells), myocytes (muscle cells) and adipocytes (fat cells). Thus, they have the ability to generate cartilage, bone, muscle, tendon, ligament, fat and other connective tissues, or components thereof. Mesenchymal stem cells are characterized morphologically by a small cell body which contains a large, round nucleus with a prominent nucleolus, which is surrounded by finely dispersed chromatin particles, giving the nucleus a clear appearance. The remainder of the cell body contains a small amount of Golgi apparatus, rough endoplasmic reticulum, mitochondria and polyribosomes. The shape of the mesenchymal stem cells is generally long and thin. Mesenchymal stromal cells can be isolated from a range of tissue types, including bone marrow, muscle, fat, dental pulp, adult tissue, fetal tissue, neonatal tissue, and umbilical cord.

The term “stem cell” as used herein refers to a cell that on division faces two developmental options: the daughter cells can be identical to the original cell (self-renewal) or they may be the progenitors of more specialised cell types (differentiation). The stem cell is therefore capable of adopting one or other pathway (a further pathway exists in which one of each cell type can be formed). Stem cells are therefore cells which are not terminally differentiated and are able to produce cells of other types. Stem cells can be described in terms of the range of cell types into which they are able to differentiate, as discussed below.

“Totipotent” stem cell refers to a cell which has the potential to become any cell type in the adult body, or any cell of the extraembryonic membranes (e.g., placenta). Thus, normally, the only totipotent cells are the fertilized egg and the first 4 or so cells produced by its cleavage.

“Pluripotent” stem cells are true stem cells, with the potential to make any differentiated cell in the body. However, they cannot contribute to make the extraembryonic membranes which are derived from the trophoblast. Embryonic Stem (ES) cells are examples of pluripotent stem cells, and may be isolated from the inner cell mass (ICM) of the blastocyst, which is the stage of embryonic development when implantation occurs.

“Multipotent” stem cells are true stem cells which can only differentiate into a limited number of cell types. For example, the bone marrow contains multipotent stem cells that give rise to all the cells of the blood but not to other types of cells. Multipotent stem cells are found in adult animals, and are sometimes called adult stem cells. It is thought that every organ in the body (brain, liver) contains them where they can replace dead or damaged cells.

The term “induced pluripotent stem cell” as used herein refers to a type of pluripotent stem cell artificially derived from a non-pluripotent cell, typically an adult somatic cell, for example fibroblasts, lung or B cells, by inserting certain genes. Induced pluripotent stem cells are typically derived by transfection of certain stem cell-associated genes into non-pluripotent cells, such as adult fibroblasts.

The term “logarithmic phase” or “log phase” in short as used herein refers to a period of cell growth characterized by cell doubling. The number of cells appearing per unit time is proportional to the present population. If growth is not limited, doubling will continue at a constant rate so both the number of cells and the rate of population increase doubles with each consecutive time period. For this type of exponential growth, plotting the natural logarithm of cell number against time produces a straight line. The slope of this line is the specific growth rate of the cell, which is a measure of the number of divisions per cell per unit time. The actual rate of this growth depends upon the growth conditions, which affect the frequency of cell division events and the probability of both daughter cells surviving. Exponential growth cannot continue indefinitely, however, because the medium is soon depleted of nutrients and enriched with wastes. The term “mid-logarithmic phase” or “mid-log phase” in short as used herein refers to the period of cell growth represented by the mid-point (i.e. about 50%) on the curve representing the log phase of growth in the cell growth plot (i.e. plotting cell number against cell culturing time). The rate of cell growth is the highest at the mid-log phase. The term “early-logarithmic phase” or “early-log phase” in short as used herein refers to the log phase before the mid-log phase. Similarly, the term “late-logarithmic phase” or “late-log phase” in short as used herein refers to the log phase after the mid-log phase.

### DETAILED DESCRIPTION OF THE PRESENT INVENTION

It has been surprisingly found by the inventors of the present invention that a critically defined combination of stem cells attached on LPCL microcarriers can achieve efficient cell growth and cartilage differentiation in vitro, and effective cartilage generation and healing in vivo.

Thus, in one aspect, there is provided a method of manufacturing an implantable construct comprising chondrogenically differentiated cells and one or more polycaprolactone (PCL) microcarriers, the method comprises: a) culturing mesenchymal stromal cells with one or more PCL microcarriers in a suspension culture in a mesenchymal stromal cells growth medium to allow the mesenchymal stromal cells to attach to the PCL microcarriers to form one or more mesenchymal stromal cells-PCL microcarrier complexes, wherein the suspension culture is agitated; b) harvesting the one or more mesenchymal stromal cells-PCL microcarrier complexes from the suspension culture in a) while the suspension culture is agitated; c) culturing the one or more mesenchymal stromal cells-PCL microcarrier complexes from b) under agitation-free and centrifugation-free conditions in the mesenchymal stromal cells growth medium; d) culturing the one or more mesenchymal stromal cells-PCL microcarrier complexes from c) under agitation-free and centrifugation-free conditions in a chondrogenic differentiation medium to enact differentiation of the mesenchymal stromal cells into chondrogenically differentiated cells. In another aspect, there is provided an implantable construct comprising chondrogenically differentiated cells and one or more PCL microcarriers, produced using the method as disclosed herein.

“Agitation” refers to stirring or disturbance of a liquid, in particular a cell culture containing liquid. Forms of agitation include but are not limited to, shaking, stirring, beating, churning, whisking, whipping, blending, rolling, and jolting of the liquid or the container containing the liquid. “Agitation-free” means no agitation is used during the particular culturing step. Similarly, “centrifugation-free” means no centrifugation is used during the particular culturing step.

In some examples, the PCL microcarriers used in the present invention to manufacture the implantable construct having chondrogenically differentiated cells and one or more PCL microcarriers are low density PCL microcarriers, i.e. PCL microcarriers with overall density of lower than 1.145 g/cm³ under standard conditions, due to the presence of inner pores in the microcarriers. In some examples, the overall density of the PCL microcarriers used is higher than its surrounding fluid. In some examples, each of the PCL microcarriers described in the present application has a density of about 1.01 to about 1.09 g/cm³, or about 1.02 to about 1.08 g/cm³, or about 1.03 to about 1.07 g/cm³, or about 1.04 to about 1.06 g/cm³, or about 1.05 to about 1.06 g/cm³. In some specific examples, each of the PCL microcarriers described in the present application has a density of about 1.05 to about 1.07 g/cm³. In one specific example, each of the PCL microcarriers described in the present application has a density of about 1.06 g/cm³.

In some examples, the PCL microcarriers used in the present invention can be characterized by the specific gravity of the PCL microcarrier with reference to its surrounding fluid. The term “specific gravity” as used herein refers to the ratio of the density of the PCL microcarrier to the density of a reference substance such as the fluid surrounding the PCL microcarrier. This term is also used to refer to the buoyancy of the microcarrier in its surrounding fluid, or the average density of the microcarrier in its surrounding fluid. When the volume of a PCL microcarrier is considered as a whole (i.e. the volume of the entire PCL microcarrier, including the volume of its inner pores, which are putatively filled with the surrounding fluid), then the “density” value of the PCL microcarrier corresponds to the specific gravity of the individual microcarrier in its surrounding fluid (e.g. the cell culture medium). For this purpose, the cell culture medium may be considered as having a density equal to that of water at 4° C., i.e. 1 g/cm³. Defining the density of the PCL material as d, total volume of a microcarrier as X (this includes the volume of PCL material, as well as the volume of the pores within the PCL microcarrier), and the total volume of the pores within the PCL microcarrier as Y, and assuming that the density of the cell culture medium is 1 g/cm³, the specific gravity of the PCL microcarrier filled with the cell culture medium can be calculated using the following formula: d−(d−1)×Y/X. Y/X is also referred to as the porosity of the PCL microcarrier. For example, when the density of the PCL material is 1.14 g/cm³, and porosity of the PCL microcarrier (i.e. Y/X) is 50%, the specific gravity of the PCL microcarrier in the cell culture medium is 1.14−(1.14−1)×50%=1.07 g/cm³.

In some examples, each of the one or more PCL microcarriers described in the present application has a mean diameter of about 50 to about 1000 μm, or about 60 to about 950 μm, or about 70 to about 900 μm, or about 80 to about 850 μm, or about 90 to about 800 μm, or about 100 to about 750 μm, or about 110 to about 700 μm, or about 120 to about 650 μm, or about 130 to about 600 μm, or about 140 to about 550 μm, or about 150 to about 500 μm, or about 160 to about 480 μm, or about 170 to about 460 μm, or about 180 to about 440 μm, or about 190 to about 420 μm, or about 200 to about 400 μm, or about 210 to about 380 μm, or about 220 to about 360 μm, or about 240 to about 340 μm, or about 260 to about 320 μm, or about 280 to about 300 μm, or at about 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 505, 525, 545, 565, 585, 605, 625, 645, 665, 685, 705, 725, 745, 765, 785, 805, 825, 845, 865, 885, 905, 925, 945, 965 or 985 μm, and a coefficient of variation (CV) of the diameter of less than 20%, or less than 18%, or less than 16%, or less than 14%, or less than 12%, or less than 10%, or less than 9%, or less than 8%, or less than 7%, or less than 6%, or less than 5%, or less than 4%, or less than 3%, or less than 2%, or less than 1%.

In one specific example, each of the one or more PCL microcarriers described in the present application has a mean diameter of about 50 to about 400 μm, and a coefficient of variation (CV) of the diameter of less than 10%. In another specific example, each of the one or more PCL microcarriers has a mean diameter of about 150 to about 200 μm, and a coefficient of variation (CV) of the diameter of less than 5%.

In some examples, the microcarriers are pure or near pure polycaprolactone. In other examples, the polycaprolactone may be blended with one or more other polymers, active substances or selected agents.

In some examples, the microcarriers comprise, or are manufactured from material having, at least 30% PCL, or one of at least 40%, 50%, 60%, 70%, 75%, 80%, 85%, 90%, 91%, 92%, 93%, 94%, 95%, 96%, 97%, 98%, 99% or 100% PCL.

Varying grades of PCL, including medical grade PCL, potentially composed of different molecular weight distributions, may similarly be used to fabricate microspheres.

In some examples, the microcarriers as disclosed herein have a surface area in the range about 300 to about 700 cm²/g (dry weight), or about 300 to about 600, about 300 to about 500, about 300 to about 400, about 400 to about 700, about 400 to about 600, about 400 to about 500, about 500 to about 700, about 500 to about 600, or about 300, 350, 400, 450, 500, 550, 600, 650 or 700 cm²/g (dry weight).

The number of microcarriers per gram (dry weight) may be in the range about 0.25×10⁸ to about 3.2×10⁸, or about 0.25×10⁸ to 3×10⁸, about 0.25×10⁸ to 2.5×10⁸, about 0.25×10⁸ to 2×10⁸, about 0.25×10⁸ to 1.5×10⁸, about 0.25×10⁸ to 1×10⁸, about 0.25×10⁸ to 0.5×10⁸, about 0.3×10⁸ to 3×10⁸, about 0.3×10⁸ to 2.5×10⁸, about 0.3×10⁸ to 2×10⁸, about 0.3×10⁸ to 1.5×10⁸, about 0.3×10⁸ to 1×10⁸, about 0.3×10⁸ to 0.5×10⁸, about 0.4×10⁸ to 3×10⁸, about 0.4×10⁸ to 2.5×10⁸, about 0.4×10⁸ to 2×10⁸, about 0.4×10⁸ to 1.5×10⁸, about 0.4×10⁸ to 1×10⁸, about 0.4×10⁸ to 0.5×10⁸, about 0.5×10⁸ to 3×10⁸, about 0.5×10⁸ to 2.5×10⁸, about 0.5×10⁸ to 2×10⁸, about 0.5×10⁸ to 1.5×10⁸, about 0.5×10⁸ to 1×10⁸, about 0.75×10⁸ to 3×10⁸, about 0.75×10⁸ to 2.5×10⁸, about 0.75×10⁸ to 2×10⁸, about 0.75×10⁸ to 1.5×10⁸, about 0.75×10⁸ to 1×10⁸, about 1×10⁸ to 3×10⁸, about 1×10⁸ to 2.5×10⁸, about 1×10⁸ to 2×10⁸, about 1×10⁸ to 1.5×10⁸, about 1.5×10⁸ to 3×10⁸, about 1.5×10⁸ to 2.5×10⁸, about 1.5×10⁸ to 2×10⁸, about 2×10⁸ to 3×10⁸, about 2×10⁸ to 2.5×10⁸, about 2.5×10⁸ to 3×10⁸. In some specific examples, the number of microcarriers per gram (dry weight) is about 0.25×10⁸ to about 1.0×10⁸. In some examples, the number of microcarriers per gram (dry weight) is about 0.25×10⁸, 0.5×10⁸, 0.75×10⁸, 1.0×10⁸, 1.25×10⁸, 1.5×10⁸, 1.75×10⁸, 2.0×10⁸, 2.25×10⁸, 2.5×10⁸, 2.75×10⁸, or 3.0×10⁸.

In some examples, the microcarriers comprise a positive charge at for example neutral pH or physiologically relevant pH such as pH 7.4 or pH 7.2. The quantity of positive charge may vary, but in some examples is intended to be high enough to enable cells to attach to the particle. For example, where the particles are charged by coupling with amines, such as quaternary or tertiary amines, the charge on the particle may correspond to a small ion exchange capacity of about 0.5 to 4 milli-equivalents per gram dry material (of the particle), for example between about 1 to 3.5 milli-equivalents per gram dry material (of the particle) or between about 1 to 2 milli-equivalents per gram dry material (of the particle). In some examples, the positive charge is such that that the pKa of the particle is greater than 7 (e.g., greater than 7.4, e.g., 7.5, 8, 8.5, 9, 9.5, 10, 10.5, 11, 11.5 or more).

In some examples, the microcarriers are derivatised by coupling for example to protamine sulphate or poly-L-lysine hydrobromide at a concentration of up to 20 mg/ml particles.

In some examples, the presence of a positive charge on the microcarriers assists attachment of cells thereto.

In some examples, the microcarriers are derivatised to carry positive charges. In some examples, the microcarriers comprise amine groups attached thereto. The amine groups can be primary amine groups, secondary amine groups, tertiary amine groups or quaternary amine groups. The amine groups can be attached to the microcarriers by coupling the microcarriers with amine containing compounds. Methods of coupling are well known in the art. For example, the amine can be coupled to the microcarriers by the use of cyanogen bromide.

Crosslinkers can also be used. These are divided into homobifunctional crosslinkers, containing two identical reactive groups, or heterobifunctional crosslinkers, with two different reactive groups. Heterobifunctional crosslinkers allow sequential conjugations, minimizing polymerization. Coupling and crosslinking reagents can be obtained from a number of manufacturers, for example, from Calbiochem or Pierce Chemical Company.

The microcarriers may be activated prior to coupling, to increase its reactivity. The compact microcarriers may be activated using chloroacetic acid followed by coupling using EDAC/NHS-OH. Microcarriers may also be activated using hexane di isocyanate to give a primary amino group. Such activated microcarriers may be used in combination with any heterobifunctional cross linker. The compact microcarriers in certain examples is activated using divinyl sulfon. Such activated compact microcarriers comprise moieties which can react with amino or thiol groups, on a peptide, for example.

The microcarriers can also be activated using tresyl chloride, giving moieties which are capable of reacting with amino or thiol groups. The microcarriers can also be activated using cyanogen chloride, giving moieties which can react with amino or thiol groups.

In some examples, the number of PCL microcarriers in the implantable construct is about 500 to about 5000, or about 600 to about 4800, or about 700 to about 4600, or about 800 to about 4400, or about 900 to about 4200, or about 1000 to about 4000, or about 1100 to about 3800, or about 1200 to about 3600, or about 1300 to about 3400, or about 1400 to about 3200, or about 1500 to about 3000, or about 1600 to about 2900, or about 1700 to about 2800, or about 1800 to about 2700, or about 1900 to about 2600, or about 2000 to about 2500, or about 2100 to about 2400, or about 2200 to about 2300, or at about 1050, 1150, 1250, 1350, 1450, 1550, 1650, 1750, 1850, 1950, 2050, 2150, 2250, 2350, 2450, 2550, 2650, 2750, 2850, 2950, 3050, 3150, 3250, 3350, 3450, 3550, 3650, 3750, 3850, 3950, 4050, 4150, 4250, 4350, 4450, 4550, 4650, 4750, 4850 or 4950. In one specific example, the number of PCL microcarriers in the implantable construct is about 2000 to about 3000.

In some examples, the ratio of the number of mesenchymal stromal cells to be cultured and the number of PCL microcarriers in a) is about 10 to about 50, or about 15 to about 45, or about 20 to about 40, or about 25 to about 35, or at about 12, 14, 16, 18, 20, 22, 24, 26, 28, 30, 32, 34, 36, 38, 40, 42, 44, 46, 48, or 50. In some specific examples, the ratio of the number of mesenchymal stromal cells to be cultured and the number of PCL microcarriers in a) is about 10 to about 30.

In some examples, the PCL microcarriers are LPCL microcarriers. In some examples, each of the one or more PCL microcarriers described in the present application is porous, hollow or a combination thereof. In some examples, each of the one or more PCL microcarriers described in the present application is of spherical, ellipsoidal, cylindrical or disc shape.

In some examples, each of the one or more PCL microcarriers described in the present application comprises a coating comprising an adhesion promoting polypeptide, a cell growth promoting polypeptide, a migration promoting polypeptide, glycopolypeptide, cationic polyelectrolyte or polysaccharide. In some examples, each of the one or more PCL microcarriers further comprises a multilayer coating comprising (i) a first layer, comprising a matrix component; and (ii) one or more other layer, each layer comprising a matrix component; wherein the matrix component is any one or more of poly-L-lysine (PLL), laminin, gelatin, collagen, keratin, fibronectin, vitronectin, hyaluronic acid, elastin, heparan sulphate, dextran, dextran sulphate, chondroitin sulphate, and a mixture of laminin, collagen I, heparan sulfate proteoglycans, entactin 1, cationic polyelectrolyte, and other implantable or resorbable polymer such as polyamides and polyacrylamides. In some specific examples, each of the one or more PCL microcarriers comprises a multilayered coating comprising a first fibronectin layer, a poly-L-lysine layer, and a second fibronectin layer.

In some examples, the mesenchymal stromal cells are obtained from embryonic, fetal or adult tissue of mammalian species. Examples of mammalian species include but are not limited to mouse, rat, rabbit, guinea pig, dog, cat, pig, sheep, cow, horse, monkey and human. In some examples, the mesenchymal stromal cells are not obtained from embryonic tissue of human origin. In some other examples, the mesenchymal stromal cells are obtained from embryonic, fetal or adult tissue of human origin. In some other examples, the mesenchymal stromal cells are not obtained from embryonic tissue of human origin harvested later than 14 days after fertilization.

In some examples, the number of mesenchymal stromal cells to be cultured in step a) of the method above is about 3×10⁴ to about 7×10⁴, or about 3.5×10⁴ to about 6.5×10⁴, or about 4×10⁴ to about 6×10⁴, or about 4.5×10⁴ to about 5.5×10⁴, or at about 3×10⁴, 3.25×10⁴, 3.5×10⁴, 3.75×10⁴, 4×10⁴, 4.25×10⁴, 4.75×10⁴, 5×10⁴, 5.25×10⁴, 5.5×10⁴, 5.75×10⁴, 6×10⁴, 6.25×10⁴, 6.5×10⁴, 6.75×10⁴ or 7×10⁴ per construct of PCL microcarriers. In some specific examples, the number of mesenchymal stromal cells to be cultured in step a) of the method above 4.5×10⁴ to about 5.5×10⁴ per construct of PCL microcarriers. In one specific example, the number of mesenchymal stromal cells to be cultured in step a) of the method above is about 5×10⁴ per construct of PCL microcarriers.

In some examples, culturing mesenchymal stromal cells with one or more PCL microcarriers in a suspension culture in step a) of the method above comprises culturing under an agitation rate of about 20 to about 60 rpm, or about 25 to about 55 rpm, or about 30 to about 50 rpm, or about 35 to about 45 rpm, or at about 30, 32, 34, 36, 38, 40, 42, 44, 46, 48 or 50 rpm. In some specific examples, culturing mesenchymal stromal cells with one or more PCL microcarriers in a suspension culture in step a) of the method above comprises culturing under an agitation rate of about 30 to about 50 rpm.

In some examples, step b) of the method above is carried out during the early log phase of step a). In some examples, the early log phase of step a) means about 1 to about 5 days, or about 2 to about 4 days, or about 2 to about 3 days, or about 1, 2, 3, 4 or 5 days, or about 24 to about 120 hours, or about 36 to about 108 hours, or about 48 to 96 hours, or about 60 to about 84 hours, or about 24, 30, 36, 42, 48, 54, 60, 66, 72, 78, 84, 90, 96, 102, 108, 114 or 120 hours from starting the culturing in step a). In some specific examples, the early log phage of step a) is about 2.5 to about 3.5 days from starting the culturing in step a). In one specific example, the early log phage of step a) is about 3 days from starting the culturing in step a). In some examples, the confluency of mesenchymal stromal cells on the PCL microcarriers at the early log phase is about 10% to about 50%, or about 15% to about 45%, or about 20% to about 40%, or about 25% to about 35%, or at about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%. In some specific examples, the confluency of mesenchymal stromal cells on the PCL microcarriers at the early log phase is at about 20% to about 30%. In one specific example, the confluency of mesenchymal stromal cells on the PCL microcarriers at the early log phase is at about 21%.

Harvesting the one or more mesenchymal stromal cells-PCL microcarrier complexes from the suspension culture in a) does not involve the dissociation of the mesenchymal stromal cells from the one or more PCL microcarriers (using for example mechanical or enzymatic methods).

In some examples, step c) and/or step d) of the method above comprises culturing the one or more mesenchymal stromal cell-microcarrier complexes in an adherent culture on a support surface. “Adherent culture” refers to the type of cell culture that requires a surface or an artificial substrate for the cells to grow on. In some examples, the support surface is a surface of a cell culture vessel, which can be a tissue slide, a microscope slide, a flask, a plate, a multi-well plate, a bottle, a bioreactor, a two or three-dimensional scaffold, a tube, a suture, a membrane or a film. In some examples, the support surface is a low adhesion support surface.

In some examples, step c) of the method above comprises culturing the one or more mesenchymal stromal cell-microcarrier complexes for about 1 day (i.e. about 24 hours), or about 6 to 36 hours, or about 12 to 30 hours, or about 18 to 24 hours.

In some examples, step d) of the method above comprises culturing the one or more mesenchymal stromal cell-microcarrier complexes from step c) for about 1 to about 28 days, or about 2 to about 27 days, or about 3 to about 26 days, or about 4 to about 25 days, or about 5 to about 24 days, or about 6 to about 23 days, or about 7 to about 22 days, or about 8 to about 21 days, or about 9 to about 20 days, or about 10 to about 19 days, or about 11 to about 18 days, or about 12 to about 17 days, or about 13 to about 16 days, or about 14 to about 15 days, or for about 1, 2, 3, 4, 5, 6, 7, 8, 9, 10, 11, 12, 13, 14, 15, 16, 17, 18, 19, 20, 21, 22, 23, 24, 25, 26, 27 or 28 days. In some specific examples, step d) comprises culturing the one or more mesenchymal stromal cell-microcarrier complexes from step c) for about 14 days to about 28 days, or for about 21 days to about 28 days. In one specific example, step d) comprises culturing the one or more mesenchymal stromal cell-microcarrier complexes from step c) for about 28 days.

In some examples, the mesenchymal stromal cells growth medium comprises a first basal medium and one or more cell culture supplements. In one example, the first basal medium is minimum essential medium a. In some examples, the one or more cell culture supplements are serum and/or antibiotic. In some examples, the concentration of the serum is at about 5%, about 6%, about 7%, about 8%, about 9%, or about 10% vol/vol. In some specific examples, the concentration of the serum is at about 8% to about 10%. In one specific example, the concentration of the serum is at about 10% vol/vol.

In some examples, the chondrogenic differentiation medium comprises a second basal medium and one or more cell culture supplements. In one example, the second basal medium is Dulbecco's Modified Eagle Medium (DMEM)-high glucose. In some examples, the one or more cell culture supplements is a TGF beta superfamily ligand, a WNT inhibitor or antagonist, a carbon supplement, a glucocorticoid pathway activator, Vitamin C or derivative thereof, a promoter of glucose and/or amino acid uptake, an iron carrier, an antioxidant, an amino acid or an antibiotic.

In some examples, the antibiotic used in the mesenchymal stromal cells growth medium and/or the chondrogenic differentiation medium is ampicillin, penicillin, chloramphenicol, gentamycin, kanamycin, neomycin, streptomycin, tetracycline, polymyxin B, actinomycin, bleomycin, cyclohexamide, geneticin (G148), hygromycin B, mitomycin C or combinations thereof. In some specific examples, the antibiotic is penicillin/streptomycin.

In some examples, the concentration of the antibiotic is about 0.1% to about 10%, or about 0.5% to about 9.5%, or about 1% to about 9%, or about 1.5% to about 8.5%, or about 2% to about 8%, or about 2.5% to about 7.5%, or about 3% to about 7%, or about 3.5% to about 6.5%, or about 4% to about 6%, or about 4.5% to about 5.5%, or at about 0.5%, 1%, 2%, 3%, 4%, 5%, 6%, 7%, 8%, 9%, or 10% vol/vol. In some specific examples, the concentration of the antibiotic is about 1% to about 2%.

In some examples, the TGF beta superfamily ligand is a bone morphogenetic protein (BMP) or a TGFβ. Examples of bone morphogenetic protein (BMP) include but are not limited to BMP1, BMP2, BMP3, BMP4, BMP5, BMP6, BMP7, BMP8a, BMP8b, BMP10 and BMP15. In one specific example, the bone morphogenetic protein (BMP) is BMP2, preferably human recombinant BMP2. Examples of TGFβ include but are not limited to TGFβ1 and TGFβ3.

In some examples, the concentration of the bone morphogenetic protein (BMP) is about 1 to about 200 ng/ml, or about 5 to about 190 ng/ml, or about 10 to about 180 ng/ml, or about 20 to about 170 ng/ml, or about 30 to about 160 ng/ml, or about 40 to about 150 ng/ml, or about 50 to about 140 ng/ml, or about 60 to about 130 ng/ml, or about 70 to about 120 ng/ml, or about 80 to about 110 ng/ml, or about 90 to about 100 ng/ml, or at about 5, 10, 15, 20, 25, 30, 35, 40, 45, 50, 55, 60, 65, 70, 75, 80, 85, 90, 95, 100, 110, 120, 130, 140, 150, 160, 170, 180, 190 or 200 ng/ml. In some specific examples, the concentration of the bone morphogenetic protein (BMP) is about 75 to about 150 ng/ml.

In some examples, the concentration of the TGFβ is about 0.5 to about 200 ng/ml, or about 5 to about 190 ng/ml, or about 10 to about 180 ng/ml, or about 20 to about 170 ng/ml, or about 30 to about 160 ng/ml, or about 40 to about 150 ng/ml, or about 50 to about 140 ng/ml, or about 60 to about 130 ng/ml, or about 70 to about 120 ng/ml, or about 80 to about 110 ng/ml, or about 90 to about 100 ng/ml, or at about 0.5, 1, 5, 10, 15, 20, 25, 30, 35, 40, 45, 50, 55, 60, 65, 70, 75, 80, 85, 90, 95, 100, 110, 120, 130, 140, 150, 160, 170, 180, 190 or 200 ng/ml. In some specific examples, the concentration of the TGFβ is about 5 to about 15 ng/ml.

In some examples, the WNT inhibitor or antagonist is Dickkopf-related protein (DKK) or secreted Frizzled-Related Protein (sFRP). Examples of DKK include but are not limited to DKK-1, DKK-2, DKK-3 and DKK-4. Examples of sFRP include but are not limited to sFRP1, sFRP2, sFRP3, sFRP4 and sFRP5.

In some examples, the concentration of the WNT inhibitor or antagonist is about 10 to about 6000 ng/ml, or about 20 to about 5500 ng/ml, or about 30 to about 5000 ng/ml, or about 40 to about 4500 ng/ml, or about 50 to about 4000 ng/ml, or about 60 to about 3500 ng/ml, or about 70 to about 3000 ng/ml, or about 80 to about 2500 ng/ml, or about 90 to about 2000 ng/ml, or about 110 to about 1500 ng/ml, or about 120 to about 1000 ng/ml, or about 130 to about 900 ng/ml, or about 140 to about 800 ng/ml, or about 150 to about 700 ng/ml, or about 160 to about 600 ng/ml, or about 170 to about 500 ng/ml, or about 180 to about 450 ng/ml, or about 190 to about 400 ng/ml, or about 200 to about 380 ng/ml, or about 210 to about 360 ng/ml, or about 220 to about 340 ng/ml, or about 230 to about 320 ng/ml, or about 240 to about 300 ng/ml, or about 250 to about 290 ng/ml, or about 260 to about 280 ng/ml, or at about 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5200, 5400, 5600, 5800 or 6000 ng/ml. In some specific examples, the concentration of the WNT inhibitor or antagonist is about 100 to about 300 ng/ml.

In some examples, the carbon supplement is sodium pyruvate. In some examples, the concentration of the carbon supplement is about 100 μM to about 10 mM, or about 200 μM to about 9.5 mM, or about 300 μM to about 9 mM, or about 400 μM to about 8.5 mM, or about 500 μM to about 8 mM, or about 600 μM to about 7.5 mM, or about 700 μM to about 7 mM, or about 800 μM to about 6.5 mM, or about 900 μM to about 6 mM, or about 1 mM to about 5.5 mM, or about 1.5 mM to about 5 mM, or about 2 mM to about 4.5 mM, or about 2.5 mM to about 4 mM, or about 3 mM to about 3.5 mM, or at about 500, 750 μM, or at about 1, 2, 3, 4, 5, 6, 7, 8, 9, 10 mM. In some specific examples, the concentration of the carbon supplement is about 0.5 to about 2 mM.

In some examples, the glucocorticoid pathway activator is dexamethasone. In some examples, the concentration of the glucocorticoid pathway activator is about 10 nM to about 1 μM, or about 20 nM to about 950 nM, or about 30 nM to about 900 nM, or about 40 nM to about 850 nM, or about 50 nM to about 800 nM, or about 60 nM to about 750 nM, or about 70 nM to about 700 nM, or about 80 nM to about 650 nM, or about 90 nM to about 600 nM, or about 100 nM to about 550 nM, or about 120 nM to about 500 nM, or about 140 nM to about 450 nM, or about 160 nM to about 400 nM, or about 180 nM to about 350 nM, or about 200 nM to about 300 nM, or about 220 nM to about 250 nM, or at about 50, 100, 150, 200, 250, 300, 350, 400, 450, 500, 550, 600, 650, 700, 750, 800, 850, 900 or 950 nM or 1 μM. In some specific examples, the concentration of the glucocorticoid pathway activator is about 50 to about 150 nM.

In some examples, the Vitamin C derivative thereof is L-ascorbic acid-2-phosphate. In some examples, the concentration of the Vitamin C or derivative thereof is about 10 μM to about 1 mM, or about 20 μM to about 950 μM, or about 30 μM to about 900 μM, or about 40 μM to about 850 μM, or about 50 μM to about 800 μM, or about 60 μM to about 750 μM, or about 70 μM to about 700 μM, or about 80 μM to about 650 μM, or about 90 μM to about 600 μM, or about 100 μM to about 550 μM, or about 120 μM to about 500 μM, or about 140 μM to about 450 μM, or about 160 μM to about 400 μM, or about 180 μM to about 350 μM, or about 200 μM to about 300 μM, or about 220 μM to about 250 μM, or at about 50, 100, 150, 200, 250, 300, 350, 400, 450, 500, 550, 600, 650, 700, 750, 800, 850, 900 or 950 μM or 1 mM. In some specific examples, the concentration of the Vitamin C or derivative thereof is about 0.5 to about 1 mM.

In some examples, the promoter of glucose and/or amino acid uptake is insulin, preferably human recombinant insulin. In some examples, the iron carrier is transferrin. In some examples, the antioxidant is selenous acid or sodium selenite. In some examples, the insulin, transferrin and selenous acid is provided as a mixture. In some examples, the concentration of the insulin, transferrin and selenous acid mixture is about 0.1% to about 10%, or about 0.5% to about 9.5%, or about 1% to about 9%, or about 1.5% to about 8.5%, or about 2% to about 8%, or about 2.5% to about 7.5%, or about 3% to about 7%, or about 3.5% to about 6.5%, or about 4% to about 6%, or about 4.5% to about 5.5%, or at about 0.5%, 1%, 2%, 3%, 4%, 5%, 6%, 7%, 8%, 9% or 10% vol/vol. In some specific examples, the concentration of the insulin, transferrin and selenous acid mixture is about 1 to about 5% vol/vol.

In some examples, the amino acid is proline, preferably L-proline. In some examples, the concentration of the amino acid is about 10 μg/ml to about 200 μg/ml, about 20 μg/ml to about 190 μg/ml, about 30 μg/ml to about 180 μg/ml, about 40 μg/ml to about 170 μg/ml, about 50 μg/ml to about 160 μg/ml, about 60 μg/ml to about 150 μg/ml, about 70 μg/ml to about 140 μg/ml, about 80 μg/ml to about 130 μg/ml, about 90 μg/ml to about 120 μg/ml, about 100 μg/ml to about 110 μg/ml, or at about 15, 25, 35, 45, 55, 65, 75, 85, 95, 105, 115, 125, 135, 145, 155, 165, 175, 185, or 195 μg/ml. In some specific examples, the concentration of the amino acid is about 20 to about 60 μg/ml.

In one specific example, the method of manufacturing an implantable construct comprising chondrogenically differentiated cells and one or more polycaprolactone (PCL) microcarriers as described above comprises the following steps: a) culturing about 4.5×10⁴ to about 5.5×10⁴ mesenchymal stromal cells with one construct of PCL microcarriers in a suspension culture in a mesenchymal stromal cells growth medium for about 2.5 to about 3.5 days or until the confluency of the mesenchymal stromal cells is about 20% to about 30%, to allow the mesenchymal stromal cells to attach to the PCL microcarriers to form mesenchymal stromal cells-PCL microcarrier complexes, wherein the suspension culture is agitated; b) harvesting the mesenchymal stromal cells-PCL microcarrier complexes from the suspension culture in a) while the suspension culture is agitated; c) culturing the mesenchymal stromal cells-PCL microcarrier complexes from b) under agitation-free and centrifugation-free conditions in the mesenchymal stromal cells growth medium for about 0.5 to about 1.5 days; d) culturing the mesenchymal stromal cells-PCL microcarrier complexes from c) under agitation-free and centrifugation-free conditions in a chondrogenic differentiation medium for about 14 days to about 28 days to enact differentiation of the mesenchymal stromal cells into chondrogenically differentiated cells.

In some examples of the implantable construct as described herein, the number of chondrogenically differentiated cells per construct is about 1 to about 2×10⁵, or about 10 to about 1.9×10⁵, or about 50 to about 1.8×10⁵, or about 100 to about 1.7×10⁵, or about 500 to about 1.6×10⁵, or about 1000 to about 1.5×10⁵, or about 2000 to about 1.4×10⁵, or about 3000 to about 1.3×10⁵, or about 4000 to about 1.2×10⁵, or about 5000 to about 1.1×10⁵, or about 6000 to about 1.0×10⁵, or about 7000 to about 9.5×10⁴, or about 8000 to about 9.0×10⁴, or about 9000 to about 8.5×10⁴, or about 1.0×10⁴ to about 8.0×10⁴, or about 1.5×10⁴ to about 7.5×10⁴, or about 2.0×10⁴ to about 7.0×10⁴, or about 2.5×10⁴ to about 6.5×10⁴, or about 3.0×10⁴ to about 6.0×10⁴, or about 3.5×10⁴ to about 5.5×10⁴, or about 4.0×10⁴ to about 5.0×10⁴, or at about 1, 5, 10, 20, 30, 40, 50, 60, 70, 80, 90, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 1.0×10⁴, 1.05×10⁴, 1.1×10⁴, 1.15×10⁴, 1.2×10⁴, 1.25×10⁴, 1.3×10⁴, 1.35×10⁴, 1.4×10⁴, 1.45×10⁴, 1.5×10⁴, 1.55×10⁴, 1.6×10⁴, 10.65×10⁴, 1.7×10⁴, 1.75×10⁴, 1.8×10⁴, 1.85×10⁴, 1.9×10⁴, 1.95×10⁴, 2.0×10⁴, 2.05×10⁴, 2.1×10⁴, 2.15×10⁴, 2.2×10⁴, 2.25×10⁴, 2.3×10⁴, 2.35×10⁴, 2.4×10⁴, 2.45×10⁴, 2.5×10⁴, 2.55×10⁴, 2.6×10⁴, 2.65×10⁴, 2.7×10⁴, 2.75×10⁴, 2.8×10⁴, 2.85×10⁴, 2.9×10⁴, 2.95×10⁴, 3.0×10⁴, 3.05×10⁴, 3.1×10⁴, 3.15×10⁴, 3.2×10⁴, 3.25×10⁴, 3.3×10⁴, 3.35×10⁴, 3.4×10⁴, 3.45×10⁴, 3.5×10⁴, 3.55×10⁴, 3.6×10⁴, 3.65×10⁴, 3.7×10⁴, 3.75×10⁴, 3.8×10⁴, 3.85×10⁴, 3.9×10⁴, 3.95×10⁴, 4.0×10⁴, 4.05×10⁴, 4.1×10⁴, 4.15×10⁴, 4.2×10⁴, 4.25×10⁴, 4.3×10⁴, 4.35×10⁴, 4.4×10⁴, 4.45×10⁴, 4.5×10⁴, 4.55×10⁴, 4.6×10⁴, 4.65×10⁴, 4.7×10⁴, 4.75×10⁴, 4.8×10⁴, 4.85×10⁴, 4.9×10⁴, 4.95×10⁴, 5.0×10⁴, 5.05×10⁴, 5.1×10⁴, 5.15×10⁴, 5.2×10⁴, 5.25×10⁴, 5.3×10⁴, 5.35×10⁴, 5.4×10⁴, 5.45×10⁴, 5.5×10⁴, 5.55×10⁴, 5.6×10⁴, 5.65×10⁴, 5.7×10⁴, 5.75×10⁴, 5.8×10⁴, 5.85×10⁴, 5.9×10⁴, 5.95×10⁴, 6.0×10⁴, 6.05×10⁴, 6.1×10⁴, 6.15×10⁴, 6.2×10⁴, 6.25×10⁴, 6.3×10⁴, 6.35×10⁴, 6.4×10⁴, 6.45×10⁴, 6.5×10⁴, 6.55×10⁴, 6.6×10⁴, 6.65×10⁴, 6.7×10⁴, 6.75×10⁴, 6.8×10⁴, 6.85×10⁴, 6.9×10⁴, 6.95×10⁴, 7.0×10⁴, 7.05×10⁴, 7.1×10⁴, 7.15×10⁴, 7.2×10⁴, 7.25×10⁴, 7.3×10⁴, 7.35×10⁴, 7.4×10⁴, 7.45×10⁴, 7.5×10⁴, 7.55×10⁴, 7.6×10⁴, 7.65×10⁴, 7.7×10⁴, 7.75×10⁴, 7.8×10⁴, 7.85×10⁴, 7.9×10⁴, 7.95×10⁴, 8.0×10⁴, 8.05×10⁴, 8.1×10⁴, 8.15×10⁴, 8.2×10⁴, 8.25×10⁴, 8.3×10⁴, 8.35×10⁴, 8.4×10⁴, 8.45×10⁴, 8.5×10⁴, 8.55×10⁴, 8.6×10⁴, 8.65×10⁴, 8.7×10⁴, 8.75×10⁴, 8.8×10⁴, 8.85×10⁴, 8.9×10⁴, 8.95×10⁴, 9.0×10⁴, 9.05×10⁴, 9.1×10⁴, 9.15×10⁴, 9.2×10⁴, 9.25×10⁴, 9.3×10⁴, 9.35×10⁴, 9.4×10⁴, 9.45×10⁴, 9.5×10⁴, 9.55×10⁴, 9.6×10⁴, 9.65×10⁴, 9.7×10⁴, 9.75×10⁴, 9.8×10⁴, 9.85×10⁴, 9.9×10⁴, 9.95×10⁴, 1.0×10⁵, 1.05×10⁵, 1.1×10⁵, 1.15×10⁵, 1.2×10⁵, 1.25×10⁵, 1.3×10⁵, 1.35×10⁵, 1.4×10⁵, 1.45×10⁵, 1.5×10⁵, 1.55×10⁵, 1.6×10⁵, 1.65×10⁵, 1.7×10⁵, 1.75×10⁵, 1.8×10⁵, 1.85×10⁵, 1.9×10⁵, 1.95×10⁵ or 2.0×10⁵. In some specific examples, the number of chondrogenically differentiated cells per construct is about 0.1×10⁵ to about 1×10⁵.

In another aspect, there is provided an implantable construct comprising chondrogenically differentiated cells and one or more PCL microcarriers, wherein the number of chondrogenically differentiated cells per PCL microcarrier is about 10 to about 30. Such implantable construct can be produced using methods described in the present application, but can also be produced using other applicable methods.

In some examples of the implantable construct as described herein, the DNA content per construct is about 0.1 to about 2.0 μg, or about 0.2 to about 1.9 μg, or about 0.3 to about 1.8 μg, or about 0.4 to about 1.7 μg, or about 0.5 to about 1.6 μg, or about 0.6 to about 1.5 μg, or about 0.7 to about 1.4 μg, or about 0.8 to about 1.3 μg, or about 0.9 to about 1.2 μg, or about 1.0 to about 1.1 μg, or at about 0.15, 0.25, 0.35, 0.45, 0.55, 0.65, 0.75, 0.85, 0.95, 1.05, 1.15, 1.25, 1.35, 1.45, 1.55, 1.65, 1.75, 1.85 or 1.95 μg. In some specific examples, the DNA content per construct is about 0.5 to about 2.0 μg, or about 0.5 to about 1.5 μg, or about 0.5 to about 1.0 μg.

In some examples of the implantable construct as described herein, the Glycosaminoglycan (GAG) content per construct is about 2 to about 120 μg, or about 3 to about 110 μg, or about 4 to about 100 μg, or about 5 to about 95 μg, or about 6 to about 90 μg, or about 7 to about 85 μg, or about 8 to about 80 μg, or about 9 to about 75 μg, or about 10 to about 70 μg, or about 12 to about 65 μg, or about 14 to about 60 μg, or about 16 to about 55 μg, or about 18 to about 50 μg, or about 20 to about 45 μg, or about 25 to about 40 μg, or about 30 to about 35 μg, or at about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118 or 120 μg. In some specific examples, the GAG content per construct is about 15 to about 50 μg.

In some examples, the GAG/DNA ratio is about 5 to about 120, or about 6 to about 115, or about 7 to about 110, or about 8 to about 105, or about 9 to about 100, or about 10 to about 95, or about 15 to about 90, or about 20 to about 85, or about 25 to about 80, or about 30 to about 75, or about 35 to about 70, or about 40 to about 65, or about 45 to about 60, or about 50 to about 55, or at about 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118 or 120. In some specific examples, the GAG/DNA ratio is about 25 to about 50.

In some examples of the implantable construct as described herein, the collagen II content per construct is about 4 to about 1400 ng, or about 5 to about 1350 ng, or about 6 to about 1300 ng, or about 7 to about 1250 ng, or about 8 to about 1200 ng, or about 9 to about 1150 ng, or about 10 to about 1100 ng, or about 15 to about 1050 ng, or about 20 to about 1000 ng, or about 25 to about 950 ng, or about 30 to about 900 ng, or about 35 to about 850 ng, or about 40 to about 800 ng, or about 45 to about 750 ng, or about 50 to about 700 ng, or about 55 to about 650 ng, or about 60 to about 600 ng, or about 65 to about 550 ng, or about 70 to about 500 ng, or about 75 to about 450 ng, or about 80 to about 400 ng, or about 85 to about 350 ng, or about 90 to about 300 ng, or about 95 to about 250 ng, or about 100 to about 150 ng, or at about 4, 6, 8, 10, 12, 14, 16, 18, 20, 22, 24, 26, 28, 30, 32, 34, 36, 38, 40, 42, 44, 46, 48, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375 or 1400 ng. In some specific examples, the collagen II content per construct is about 150 to about 500 ng.

In some examples, the collagen II/DNA ratio is about 5 to about 2000, or about 10 to about 1900, or about 20 to about 1800, or about 30 to about 1700, or about 40 to about 1600, or about 50 to about 1500, or about 60 to about 1400, or about 70 to about 1300, or about 80 to about 1200, or about 90 to about 1100, or about 100 to about 1000, or about 150 to about 950, or about 200 to about 900, or about 250 to about 850, or about 300 to about 800, or about 350 to about 750, or about 400 to about 700, or about 450 to about 650, or about 500 to about 600, or at about 5, 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1050, 1100, 1150, 1200, 1250, 1300, 1350, 1400, 1450, 1500, 1550, 1600, 1650, 1700, 1750, 1800, 1850, 1900, 1950 or 2000. In some specific examples, the collagen II/DNA ratio is about 200 to about 500.

In another aspect, there is provided a method of treating a disease or disorder associated with cartilage defect, the method comprises administering the implantable construct as described above in a patient suffering from the disease or disorder. In some examples, there is provided the implantable construct as described above for use in treating a disease or disorder associated with cartilage defect. In some other examples, there is provided use of the implantable construct as described above in the manufacture of a medicament for the treatment of a disease or disorder associated with cartilage defect.

In some examples, the disease or disorder is selected from the group consisting of osteoarthritis (OA), rheumatoid arthritis (RA), osteochondroma, cartilage injury and sports injury.

In one aspect, there is provided a method of promoting cartilage tissue regeneration in a patient in need thereof, the method comprises administering the implantable construct of the present invention to the patient. In some examples, there is provided the implantable construct of the present invention for use in promoting cartilage tissue regeneration in a patient in need thereof. In some other examples, there is provided use of the implantable construct of the present invention in the manufacture of a medicament for the promotion of cartilage tissue regeneration in a patient in need thereof.

In some examples, the implantable construct is administered to the patient via injection, surgery or transplantation.

In some examples, the method comprises autologous administration, or allogeneic administration, or xerographic administration of the composition or the implantable device.

In some examples, administering the implantable construct comprises administering about 40 to about 400, or about 50 to about 390, or about 60 to about 380, or about 70 to about 370, or about 80 to about 360, or about 90 to about 350, or about 100 to about 340, or about 110 to about 330, or about 120 to about 320, or about 130 to about 310, or about 140 to about 300, or about 150 to about 290, or about 160 to about 280, or about 170 to about 270, or about 180 to about 260, or about 190 to about 250, or about 200 to about 240, or about 210 to about 230, or at about 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395 or 400 microcarriers per mm³ of cartilage defect. In some specific examples, administering the implantable construct comprises administering about 140 to about 240, or about 145 to about 235, or about 150 to about 230, or about 155 to about 225, or about 160 to about 220, or about 165 to about 215, or about 170 to about 210, or about 175 to about 205, or about 180 to about 200, or about 185 to about 195, or at about 140, 145, 150, 155, 160, 165, 170, 175, 180, 185, 190, 195, 200, 205, 210, 215, 220, 225, 230, 235 or 240 microcarriers per mm³ of cartilage defect. In some other specific examples, administering the implantable construct comprises administering about 140 to about 150, or about 142 to about 148, or about 144 to about 146, or at about 140, 141, 142, 143, 144, 145, 146, 147, 148, 149 or 150 microcarriers per mm³ of cartilage defect. In some other specific examples, administering the implantable construct comprises administering about 140 to about 150 microcarriers per mm³ of cartilage defect.

The volume of the implantable construct to be administered, or the number of microcarriers to be administered, should not exceed the free volume of the defect. In some examples, administering the implantable construct comprises occupying about 3% to about 28%, or about 4% to about 27%, or about 5% to about 26%, or about 6% to about 25%, or about 7% to about 24%, or about 8% to about 23%, or about 9% to about 22%, or about 10% to about 21%, or about 11% to about 20%, or about 12% to about 19%, or about 13% to about 18%, or about 14% to about 17%, or about 15% to about 16%, or at about 3, 3.5, 4, 4.5, 5, 5.5, 6, 6.6, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5 or 28% of the cartilage defect. In some specific examples, administering the implantable construct comprises occupying about 10% to about 28% of the cartilage defect.

In some examples, administering the implantable construct comprises administering about 0.008 to about 1.6×10⁵, or about 0.01 to about 1.55×10⁵, or about 0.02 to about 1.5×10⁵, or about 0.03 to about 1.45×10⁵, or about 0.04 to about 1.4×10⁵, or about 0.05 to about 1.35×10⁵, or about 0.06 to about 1.3×10⁵, or about 0.07 to about 1.25×10⁵, or about 0.08 to about 1.2×10⁵, or about 0.09 to about 1.15×10⁵, or about 0.1 to about 1.1×10⁵, or about 0.2 to about 1.05×10⁵, or about 0.3 to about 1×10⁵, or about 0.4 to about 9.5×10⁴, or about 0.5 to about 9×10⁴, or about 0.6 to about 8.5×10⁴, or about 0.7 to about 8×10⁴, or about 0.8 to about 7.5×10⁴, or about 0.9 to about 7×10⁴, or about 1 to about 6.5×10⁴, or about 1.1 to about 6×10⁴, or about 1.2 to about 5.5×10⁴, or about 1.3 to about 5×10⁴, or about 1.4 to about 4.5×10⁴, or about 1.5 to about 4×10⁴, or about 1.6 to about 3.5×10⁴, or about 1.7 to about 3×10⁴, or about 1.8 to about 2.5×10⁴, or about 1.9 to about 2×10⁴, or about 2 to about 1.5×10⁴, or about 2.5 to about 1×10⁴, or about 3 to about 9500, or about 3.5 to about 9000, or about 4 to about 8500, or about 4.5 to about 8000, or about 5 to about 7500, or about 6 to about 7000, or about 7 to about 6500, or about 8 to about 6000, or about 9 to about 5500, or about 10 to about 5000, or about 15 to about 4500, or about 20 to about 4000, or about 25 to about 3500, or about 30 to about 3000, or about 35 to about 2500, or about 40 to about 2000, or about 45 to about 1500, or about 50 to about 1000, or about 60 to about 900, or about 70 to about 800, or about 80 to about 700, or about 90 to about 600, or about 100 to about 500, or about 150 to about 450, or about 200 to about 400, or about 250 to about 350, or at about 0.008, 0.01, 0.1, 1, 5, 10, 50, 100, 200, 300, 400, 500, 600, 700, 800, 900, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, 9000, 9200, 9400, 9600, 9800, 1×10⁴, 1.1×10⁴, 1.2×10⁴, 1.3×10⁴, 1.4×10⁴, 1.5×10⁴, 1.6×10⁴, 1.7×10⁴, 1.8×10⁴, 1.9×10⁴, 2×10⁴, 2.1×10⁴, 2.2×10⁴, 2.3×10⁴, 2.4×10⁴, 2.5×10⁴, 2.6×10⁴, 2.7×10⁴, 2.8×10⁴, 2.9×10⁴, 3×10⁴, 3.1×10⁴, 3.2×10⁴, 3.3×10⁴, 3.4×10⁴, 3.5×10⁴, 3.6×10⁴, 3.7×10⁴, 3.8×10⁴, 3.9×10⁴, 4×10⁴, 4.1×10⁴, 4.2×10⁴, 4.3×10⁴, 4.4×10⁴, 4.5×10⁴, 4.6×10⁴, 4.7×10⁴, 4.8×10⁴, 4.9×10⁴, 5×10⁴, 5.1×10⁴, 5.2×10⁴, 5.3×10⁴, 5.4×10⁴, 5.5×10⁴, 5.6×10⁴, 5.7×10⁴, 5.8×10⁴, 5.9×10⁴, 6×10⁴, 6.1×10⁴, 6.2×10⁴, 6.3×10⁴, 6.4×10⁴, 6.5×10⁴, 6.6×10⁴, 6.7×10⁴, 6.8×10⁴, 6.9×10⁴, 7×10⁴, 7.1×10⁴, 7.2×10⁴, 7.3×10⁴, 7.4×10⁴, 7.5×10⁴, 7.6×10⁴, 7.7×10⁴, 7.8×10⁴, 7.9×10⁴, 8×10⁴, 8.1×10⁴, 8.2×10⁴, 8.3×10⁴, 8.4×10⁴, 8.5×10⁴, 8.6×10⁴, 8.7×10⁴, 8.8×10⁴, 8.9×10⁴, 9×10⁴, 9.1×10⁴, 9.2×10⁴, 9.3×10⁴, 9.4×10⁴, 9.5×10⁴, 9.6×10⁴, 9.7×10⁴, 9.8×10⁴, 9.9×10⁴, 1×10⁵, 1.05×10⁵, 1.1×10⁵, 1.15×10⁵, 1.2×10⁵, 1.25×10⁵, 1.3×10⁵, 1.35×10⁵, 1.4×10⁵, 1.45×10⁵, 1.5×10⁵, 1.55×10⁵ or 1.6×10⁶ cells per mm³ of cartilage defect. In some specific examples, administering the implantable construct comprises administering about 2000 cells to about 6000 cells per mm³ of cartilage defect. In one specific example, administering the implantable construct comprises administering about 4000 cells per mm³ of cartilage defect.

Administering microcarriers to the area of defect involves sphere packing. A sphere packing is an arrangement of non-overlapping spheres within a containing space. In some examples, the microcarriers administered to the cartilage defect are substantially equal in size, and the packing density is about 10 to about 60%, or about 20% to about 50%, or about 30% to about 40%, or at about 15, 25, 35, 45, or 55%. In some other examples, the microcarriers administered to the cartilage defect are not equal in size, and the packing density is about 10 to about 90%, or about 20% to about 80%, or about 30% to about 70%, or about 40% to about 60%, or at about 15, 25, 35, 45, 50, 55, 65, 75 or 85%.

In another aspect, there is provided a method of treating a disease or disorder associated with cartilage defect, the method comprises administering one or more cell-free polycaprolactone (PCL) microcarriers in a patient suffering from the disease or disorder. In some examples, there is provided one or more cell-free polycaprolactone (PCL) microcarriers for use in treating a disease or disorder associated with cartilage defect. In some other examples, there is provided use of one or more cell-free polycaprolactone (PCL) microcarriers in the manufacture of a medicament for the treatment of a disease or disorder associated with cartilage defect.

In some examples, the disease or disorder is selected from the group consisting of osteoarthritis (OA), rheumatoid arthritis (RA), osteochondroma, cartilage injury and sports injury.

In another aspect, there is provided a method of promoting cartilage tissue regeneration in a patient in need thereof, the method comprises administering one or more cell-free polycaprolactone (PCL) microcarriers in the patient. In some examples, there is provided one or more cell-free polycaprolactone (PCL) microcarriers for use in promoting cartilage tissue regeneration in a patient in need thereof. In some other examples, there is provided use of one or more cell-free polycaprolactone (PCL) microcarriers in the manufacture of a medicament for the promotion of cartilage tissue regeneration in a patient in need thereof.

In some examples, the one or more polycaprolactone (PCL) microcarriers are administered to the patient via injection, surgery or transplantation.

In some examples, the method comprises autologous administration, or allogeneic administration, or xerographic administration of the composition or the implantable device.

In some examples, administering the one or more cell-free polycaprolactone (PCL) microcarriers comprises administering about 40 to about 400, or about 50 to about 390, or about 60 to about 380, or about 70 to about 370, or about 80 to about 360, or about 90 to about 350, or about 100 to about 340, or about 110 to about 330, or about 120 to about 320, or about 130 to about 310, or about 140 to about 300, or about 150 to about 290, or about 160 to about 280, or about 170 to about 270, or about 180 to about 260, or about 190 to about 250, or about 200 to about 240, or about 210 to about 230, or at about 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395 or 400 microcarriers per mm³ of cartilage defect. In some specific examples, administering the one or more cell-free polycaprolactone (PCL) microcarriers comprises administering about 140 to about 240, or about 145 to about 235, or about 150 to about 230, or about 155 to about 225, or about 160 to about 220, or about 165 to about 215, or about 170 to about 210, or about 175 to about 205, or about 180 to about 200, or about 185 to about 195, or at about 140, 145, 150, 155, 160, 165, 170, 175, 180, 185, 190, 195, 200, 205, 210, 215, 220, 225, 230, 235 or 240 microcarriers per mm³ of cartilage defect. In some other specific examples, administering the one or more cell-free polycaprolactone (PCL) microcarriers comprises administering about 140 to about 150, or about 142 to about 148, or about 144 to about 146, or at about 140, 141, 142, 143, 144, 145, 146, 147, 148, 149 or 150 microcarriers per mm³ of cartilage defect. In some other specific examples, administering the one or more cell-free polycaprolactone (PCL) microcarriers comprises administering about 140 to about 150 microcarriers per mm³ of cartilage defect.

The number of cell-free microcarriers to be administered, should not exceed the free volume of the defect. In some examples, administering the one or more cell-free polycaprolactone (PCL) microcarriers comprises occupying about 3% to about 28%, or about 4% to about 27%, or about 5% to about 26%, or about 6% to about 25%, or about 7% to about 24%, or about 8% to about 23%, or about 9% to about 22%, or about 10% to about 21%, or about 11% to about 20%, or about 12% to about 19%, or about 13% to about 18%, or about 14% to about 17%, or about 15% to about 16%, or at about 3, 3.5, 4, 4.5, 5, 5.5, 6, 6.6, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5 or 28% of the cartilage defect. In some specific examples, administering the one or more cell-free polycaprolactone (PCL) microcarriers comprises occupying about 10% to about 28% of the cartilage defect.

Administering cell-free microcarriers to the area of defect involves sphere packing. In some examples, the cell-free microcarriers administered to the cartilage defect are substantially equal in size, and the packing density is about 10 to about 60%, or about 20% to about 50%, or about 30% to about 40%, or at about 15, 25, 35, 45, or 55%. In some other examples, the cell-free microcarriers administered to the cartilage defect are not equal in size, and the packing density is about 10 to about 90%, or about 20% to about 80%, or about 30% to about 70%, or about 40% to about 60%, or at about 15, 25, 35, 45, 50, 55, 65, 75 or 85%.

In some examples, administering the implantable construct as described above results in fewer microcarrier residues within the cartilage defect as compared to administering one or more cell-free polycaprolactone (PCL) microcarriers as described above. In some examples, the number of microcarrier residues within the cartilage defect resulted from administering the implantable construct described above is about 10% to 95%, or about 20% to about 90%, or about 30% to about 80%, or about 40% to about 70%, or about 50% to about 60%, or about 10%, 15%, 20%, 25%, 30%, 35%, 40%, 45%, 50%, 55%, 60%, 65%, 70%, 75%, 80%, 85%, 90% or 95% less as compared to the number of microcarrier residues within the cartilage defect resulted from administering one or more cell-free polycaprolactone (PCL) microcarriers as described above. In some specific examples, the number of microcarrier residues within the cartilage defect resulted from administering the implantable construct described above is about 50% to 95% less as compared to the number of microcarrier residues within the cartilage defect resulted from administering one or more cell-free polycaprolactone (PCL) microcarriers as described above.

In another aspect, there is provided a method of treating a disease or disorder associated with bone defect, the method comprises administering one or more cell-free polycaprolactone (PCL) microcarriers in a patient suffering from the disease or disorder. In some examples, there is provided one or more cell-free polycaprolactone (PCL) microcarriers for use in treating a disease or disorder associated with bone defect. In some other examples, there is provided use of one or more cell-free polycaprolactone (PCL) microcarriers in the manufacture of a medicament for the treatment of a disease or disorder associated with bone defect.

In some examples, the disease or disorder is selected from the group consisting of osteoarthritis (OA), rheumatoid arthritis (RA), osteoporosis, osteogenesis imperfecta, osteochondroma, ostronecrosis, bone fracture and sports injury.

In another aspect, there is provided a method of promoting bone tissue regeneration in a patient in need thereof, the method comprises administering one or more cell-free polycaprolactone (PCL) microcarriers in the patient. In some examples, there is provided one or more cell-free polycaprolactone (PCL) microcarriers for use in promoting bone tissue regeneration in a patient in need thereof. In some other examples, there is provided use of one or more cell-free polycaprolactone (PCL) microcarriers in the manufacture of a medicament for the promotion of bone tissue regeneration in a patient in need thereof.

In some examples, the one or more polycaprolactone (PCL) microcarriers are administered to the patient via injection, surgery or transplantation.

In some examples, the method comprises autologous administration, or allogeneic administration, or xerographic administration of the composition or the implantable device.

In some examples, administering the one or more cell-free polycaprolactone (PCL) microcarriers comprises administering about 40 to about 400, or about 50 to about 390, or about 60 to about 380, or about 70 to about 370, or about 80 to about 360, or about 90 to about 350, or about 100 to about 340, or about 110 to about 330, or about 120 to about 320, or about 130 to about 310, or about 140 to about 300, or about 150 to about 290, or about 160 to about 280, or about 170 to about 270, or about 180 to about 260, or about 190 to about 250, or about 200 to about 240, or about 210 to about 230, or at about 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395 or 400 microcarriers per mm³ of bone defect. In some specific examples, administering the one or more cell-free polycaprolactone (PCL) microcarriers comprises administering about 40 to about 100, or about 45 to about 95, or about 50 to about 90, or about 55 to about 85, or about 60 to about 80, or about 65 to about 75, or at about 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100 microcarriers per mm³ of bone defect. In some specific examples, administering the one or more cell-free polycaprolactone (PCL) microcarriers comprises administering about 70 to about 90 microcarriers per mm³ of bone defect. In some other specific examples, administering the one or more cell-free polycaprolactone (PCL) microcarriers comprises administering about 80 microcarriers per mm³ of bone defect.

Administering cell-free microcarriers to the area of defect involves sphere packing. In some examples, the cell-free microcarriers administered to the bone defect are substantially equal in size, and the packing density is about 10 to about 60%, or about 20% to about 50%, or about 30% to about 40%, or at about 15, 25, 35, 45, or 55%. In some other examples, the cell-free microcarriers administered to the bone defect are not equal in size, and the packing density is about 10 to about 90%, or about 20% to about 80%, or about 30% to about 70%, or about 40% to about 60%, or at about 15, 25, 35, 45, 50, 55, 65, 75 or 85%.

In another aspect, there is provided a method of manufacturing an implantable construct comprising mesenchymal stromal cells and one or more polycaprolactone (PCL) microcarriers, the method comprises: a) culturing mesenchymal stromal cells with one or more PCL microcarriers to allow the mesenchymal stromal cells to attach to the PCL microcarriers to form mesenchymal stromal cells-PCL microcarrier complexes; b) culturing the one or more mesenchymal stromal cells-PCL microcarrier complexes from a) in a suspension culture in a mesenchymal stromal cells growth medium, wherein the suspension culture is agitated; c) harvesting the one or more mesenchymal stromal cells-PCL microcarrier complexes from the suspension culture in b) during the mid-log phase or late log phase of b) to obtain the implantable construct.

The above method of manufacturing the implantable construct comprising mesenchymal stromal cells and one or more PCL microcarriers does not involve the dissociation of the mesenchymal stromal cells from the one or more PCL microcarriers (using for example mechanical or enzymatic methods).

In some examples, step a) of the method as described above comprises culturing the mesenchymal stromal cells with the one or more PCL microcarriers in a static suspension culture in a mesenchymal stromal cells growth medium.

In another aspect, there is provided an implantable construct comprising mesenchymal stromal cells and one or more PCL microcarriers, produced using the method as described above. In another aspect, there is provided a method of treating a disease or disorder associated with bone defect, the method comprises administering the implantable construct as described above in a patient suffering from the disease or disorder. In some examples, there is provided the implantable construct as described above for use in treating a disease or disorder associated with bone defect. In some other examples, there is provided use of the implantable construct as described above in the manufacture of a medicament for the treatment of a disease or disorder associated with bone defect. Examples of the disease or disorder include but are not limited to osteoarthritis (OA), rheumatoid arthritis (RA), osteoporosis, osteogenesis imperfecta, osteochondroma, ostronecrosis, bone fracture and sports injury. In another aspect, there is provided a method of promoting bone tissue regeneration in a patient in need thereof, the method comprises administering the implantable construct as described above in the patient. In some examples, there is provided the implantable construct as described above for use in promoting bone tissue regeneration in a patient in need thereof. In some other examples, there is provided use of the implantable construct as described above in the manufacture of a medicament for the promotion of bone tissue regeneration in a patient in need thereof.

In some examples, the number of PCL microcarriers in the implantable construct is about 500 to about 5000, or about 600 to about 4800, or about 700 to about 4600, or about 800 to about 4400, or about 900 to about 4200, or about 1000 to about 4000, or about 1100 to about 3800, or about 1200 to about 3600, or about 1300 to about 3400, or about 1400 to about 3200, or about 1500 to about 3000, or about 1600 to about 2900, or about 1700 to about 2800, or about 1800 to about 2700, or about 1900 to about 2600, or about 2000 to about 2500, or about 2100 to about 2400, or about 2200 to about 2300, or at about 1050, 1150, 1250, 1350, 1450, 1550, 1650, 1750, 1850, 1950, 2050, 2150, 2250, 2350, 2450, 2550, 2650, 2750, 2850, 2950, 3050, 3150, 3250, 3350, 3450, 3550, 3650, 3750, 3850, 3950, 4050, 4150, 4250, 4350, 4450, 4550, 4650, 4750, 4850 or 4950. In some specific examples, the number of PCL microcarriers in the implantable construct is about 500 to 1500.

In some examples, the number of mesenchymal stromal cells to be cultured in step a) of the method above is about 0.1×10⁴ to about 1×10⁵, or about 0.2×10⁴ to about 9.5×10⁴, or about 0.3×10⁴ to about 9×10⁴, or about 0.4×10⁴ to about 8.5×10⁴, or about 0.5×10⁴ to about 8×10⁴, or about 0.6×10⁴ to about 7.5×10⁴, or about 0.7×10⁴ to about 7×10⁴, or about 0.8×10⁴ to about 6.5×10⁴, or about 0.9×10⁴ to about 6×10⁴, or about 1×10⁴ to about 5.5×10⁴, or about 1.1×10⁴ to about 5×10⁴, or about 1.2×10⁴ to about 4.5×10⁴, or about 1.3×10⁴ to about 4×10⁴, or about 1.4×10⁴ to about 3.5×10⁴, or about 1.5×10⁴ to about 3×10⁴, or about 1.6×10⁴ to about 2.8×10⁴, or about 1.7×10⁴ to about 2.7×10⁴, or about 1.8×10⁴ to about 2.6×10⁴, or about 1.9×10⁴ to about 2.5×10⁴, or about 2×10⁴ to about 2.4×10⁴, or about 2.1×10⁴ to about 2.3×10⁴, or at about 0.1×10⁴, 0.25×10⁴, 0.75×10⁴, 1×10⁴, 1.25×10⁴, 1.5×10⁴, 1.75×10⁴, 2×10⁴, 2.25×10⁴, 2.5×10⁴, 2.75×10⁴, 3×10⁴, 3.25×10⁴, 3.5×10⁴, 3.75×10⁴, 4×10⁴, 3.25×10⁴, 3.5×10⁴, 3.75×10⁴, 4×10⁴ 3.25×10⁴, 3.5×10⁴, 3.75×10⁴, 4×10⁴, 4.25×10⁴, 4.5×10⁴, 4.75×10⁴, 5×10⁴, 5.25×10⁴, 5.5×10⁴, 5.75×10⁴, 6×10⁴, 6.25×10⁴, 6.5×10⁴, 6.75×10⁴, 7×10⁴, 7.25×10⁴, 7.5×10⁴, 7.75×10⁴, 8×10⁴, 8.25×10⁴, 8.5×10⁴, 8.75×10⁴, 9×10⁴, 9.25×10⁴, 9.5×10⁴, 9.75×10⁴ or 1×10⁵ per construct of PCL microcarriers. In some specific examples, the number of mesenchymal stromal cells to be cultured in step a) of the method above is about 0.1×10⁴ to about 1.5×10⁴ per construct of PCL microcarriers.

In some examples, the ratio of the number of mesenchymal stromal cells to be cultured and the number of PCL microcarriers in step a) is about 3 to about 20, or about 4 to about 19, or about 5 to about 18, or about 6 to about 17, or about 7 to about 16, or about 8 to about 15, or about 9 to about 14, or about 10 to about 13, or about 11 to about 12, or at about 3, 4, 5, 6, 7, 8, 9, 10, 11, 12, 13, 14, 15, 16, 17, 18, 19 or 20. In some specific examples, the ratio is about 5 to 20.

In some examples, culturing mesenchymal stromal cells with one or more PCL microcarriers in a suspension culture in step b) comprises culturing under an agitation rate of about 20 to about 60 rpm, or about 25 to about 55 rpm, or about 30 to about 50 rpm, or about 35 to about 45 rpm, or at about 30, 32, 34, 36, 38, 40, 42, 44, 46, 48 or 50 rpm. In some specific examples, culturing mesenchymal stromal cells with one or more PCL microcarriers in a suspension culture in step b) comprises culturing under an agitation rate of about 20 to about 60 rpm. In some other specific examples, culturing mesenchymal stromal cells with one or more PCL microcarriers in a suspension culture in step b) comprises culturing under an agitation rate of about 30 to about 50 rpm. In one specific example, culturing mesenchymal stromal cells with one or more PCL microcarriers in a suspension culture in step b) comprises culturing under an agitation rate of about 40 rpm.

In some examples, the mid-log phase of step b) is about 2 to about 4 days, or about 2 to about 3 days, or about 2, 3 or 4 days, or about 48 to about 96 hours, or about 54 to about 90 hours, or about 60 to 84 hours, or about 66 to about 78 hours, or about 48, 54, 60, 66, 72, 78, 84, 90, or 96 hours from starting the culturing in step b). In some specific examples, the mid-log phage of step b) is about 2.5 to about 3.5 days from starting the culturing in step b). In some specific examples, the mid-log phage of step b) is about 3 days from starting the culturing in step b). In some examples, the confluency of mesenchymal stromal cells on the PCL microcarriers at the mid-log phase is about 40% to about 60%, or about 42% to about 58%, or about 44% to about 56%, or about 46% to about 54%, or about 48% to about 52%, or at about 40%, 41%, 42%, 43%, 44%, 45%, 46%, 47%, 48%, 49%, 50%, 51%, 52%, 53%, 54%, 55%, 56%, 57%, 58%, 59% or 60%. In some specific examples, the confluency of mesenchymal stromal cells on the PCL microcarriers at the mid-log phase is at about 45% to about 55%. In some specific examples, the confluency of mesenchymal stromal cells on the PCL microcarriers at the mid-log phase is at about 50%.

In some examples, the late log phase of step b) is about 4 to about 6 days, or about 4 to about 5 days, or about 4, 5 or 6 days, or about 96 to about 144 hours, or about 102 to about 138 hours, or about 108 to 132 hours, or about 114 to about 126 hours, or about 96, 102, 108, 114, 120, 126, 132, 138, 144 hours from starting the culturing in step b). In some specific examples, the late log phage of step b) is about 4.5 to about 5.5 days from starting the culturing in step b). In some specific examples, the late log phage of step b) is about 5 days from starting the culturing in step b). In some examples, the confluency of mesenchymal stromal cells on the PCL microcarriers at the late log phase is about 60% to about 100%, or about 62% to about 98%, or about 64% to about 96%, or about 66% to about 94%, or about 68% to about 92%, or about 70% to about 90%, or about 72% to about 88%, or about 74% to about 86%, or about 76% to about 84%, or about 78% to about 82%, or at about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%. In some examples, the confluency of mesenchymal stromal cells on the PCL microcarriers at the late log phase is about 60% to about 90%.

In some examples, the mesenchymal stromal cells growth medium comprises a first basal medium and one or more cell culture supplements.

In some examples, the first basal medium is minimum essential medium a.

In some examples, the one or more cell culture supplements are serum and/or antibiotic.

In some examples, the concentration of the serum is at about 5%, about 6%, about 7%, about 8%, about 9%, or about 10% vol/vol. In some specific examples, the concentration of the serum is at about 8% to about 10%. In some specific examples, the concentration of the serum is at about 10% vol/vol.

In some examples, the antibiotic is ampicillin, penicillin, chloramphenicol, gentamycin, kanamycin, neomycin, streptomycin, tetracycline, polymyxin B, actinomycin, bleomycin, cyclohexamide, geneticin (G148), hygromycin B, mitomycin C or combinations thereof. In some specific examples, the antibiotic is penicillin/streptomycin. In some examples, the concentration of penicillin is about 10 U/ml to about 300 U/ml, or about 20 U/ml to about 280 U/ml, or about 30 U/ml to about 260 U/ml, or about 40 U/ml to about 240 U/ml, or about 50 U/ml to about 220 U/ml, or about 60 U/ml to about 200 U/ml, or about 70 U/ml to about 180 U/ml, or about 80 U/ml to about 160 U/ml, or about 90 U/ml to about 140 U/ml, or about 100 U/ml to about 120 U/ml, or at about 15, 25, 35, 45, 55, 65, 75, 85, 95, 105, 115, 125, 135, 145, 155, 165, 175, 185, 195, 205, 215, 225, 235, 245, 255, 265, 275, 285 or 295 U/ml. In some examples, the concentration of streptomycin is about 10 mg/ml to about 300 mg/ml, or about 20 mg/ml to about 280 mg/ml, or about 30 mg/ml to about 260 mg/ml, or about 40 mg/ml to about 240 mg/ml, or about 50 mg/ml to about 220 mg/ml, or about 60 mg/ml to about 200 mg/ml, or about 70 mg/ml to about 180 mg/ml, or about 80 mg/ml to about 160 mg/ml, or about 90 mg/ml to about 140 mg/ml, or about 100 mg/ml to about 120 mg/ml, or at about 15, 25, 35, 45, 55, 65, 75, 85, 95, 105, 115, 125, 135, 145, 155, 165, 175, 185, 195, 205, 215, 225, 235, 245, 255, 265, 275, 285 or 295 mg/ml. In some specific examples, the concentration of the antibiotics is about 1% to about 2% vol/vol.

In some examples of the implantable construct as described above, the mesenchymal stromal cells produce cytokine at about 1 to about 200 pg/10⁵ cells/day, or at about 5 to about 190 pg/10⁵ cells/day, or at about 10 to about 180 pg/10⁵ cells/day, or at about 15 to about 170 pg/10⁵ cells/day, or at about 20 to about 160 pg/10⁵ cells/day, or at about 25 to about 150 pg/10⁵ cells/day, or at about 30 to about 140 pg/10⁵ cells/day, or at about 35 to about 130 pg/10⁵ cells/day, or at about 40 to about 120 pg/10⁵ cells/day, or at about 45 to about 110 pg/10⁵ cells/day, or at about 50 to about 100 pg/10⁵ cells/day, or at about 55 to about 95 pg/10⁵ cells/day, or at about 60 to about 90 pg/10⁵ cells/day, or at about 65 to about 85 pg/10⁵ cells/day, or at about 70 to about 80 pg/10⁵ cells/day, or at about 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 110, 120, 130, 140, 150, 160, 170, 180, 190 or 200 pg/10⁵ cells/day. Examples of cytokine include but are not limited to of IL6, IL8, SDF-1α, MCP-1, GRO-α and VEGF-α.

In one specific example, the method of manufacturing an implantable construct comprising mesenchymal stromal cells and one or more polycaprolactone (PCL) microcarriers as described above comprises the following steps: a) culturing about 4.5×10⁴ to about 5.5×10⁴ mesenchymal stromal cells with one construct of PCL microcarriers in a static suspension culture to allow the mesenchymal stromal cells to attach to the PCL microcarriers to form mesenchymal stromal cells-PCL microcarrier complexes; b) culturing the mesenchymal stromal cells-PCL microcarrier complexes from a) in a suspension culture in a mesenchymal stromal cells growth medium for about 2.5 to about 3.5 days or until the confluency of mesenchymal stromal cells is about 45% to 55%, wherein the suspension culture is agitated at about 30 to about 50 rpm; and c) harvesting the mesenchymal stromal cells-PCL microcarrier complexes from the suspension culture in b) to obtain the implantable construct.

In some examples, the implantable construct is administered to the patient via injection, surgery or transplantation.

In some examples, the method comprises autologous administration, or allogeneic administration, or xerographic administration of the composition or the implantable device.

In some examples, administering the implantable construct comprises administering about 40 to about 100, or about 45 to about 95, or about 50 to about 90, or about 55 to about 85, or about 60 to about 80, or about 65 to about 75, or at about 40, 42, 44, 46, 48, 50, 52, 54, 56, 58, 60, 62, 64, 66, 68, 70, 72, 74, 76, 78, 80, 82, 84, 86, 88, 90, 92, 94, 96, 98 or 100 microcarriers per mm³ of bone defect. In some specific examples, administering the implantable construct comprises administering about 70 to about 90 microcarriers per mm³ of bone defect. In some specific examples, administering the implantable construct comprises administering about 80 microcarriers per mm³ of bone defect.

In some examples, administering the implantable construct comprises administering about 100 to about 5000, or about 150 to about 4900, or about 200 to about 4800, or about 250 to about 4700, or about 300 to about 4600, or about 350 to about 4500, or about 400 to about 4400, or about 450 to about 4300, or about 500 to about 4200, or about 550 to about 4100, or about 600 to about 4000, or about 650 to about 3900, or about 700 to about 3800, or about 650 to about 3900, or about 700 to about 3800, or about 750 to about 3700, or about 800 to about 3600, or about 850 to about 3500, or about 900 to about 3400, or about 950 to about 3300, or about 1000 to about 3200, or about 1100 to about 3100, or about 1200 to about 3000, or about 1300 to about 2900, or about 1400 to about 2800, or about 1500 to about 2700, or about 1600 to about 2600, or about 1700 to about 2500, or about 1800 to about 2400, or about 1900 to about 2300, or about 2000 to about 2200, or at about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950 or 5000 cells per mm³ of bone defect. In some specific examples, administering the implantable construct comprises administering about 3000 to about 3500, or at about 3000, 3050, 3100, 3150, 3200, 3250, 3300, 3350, 3400, 3450 or 3500 cells per mm³ of bone defect.

Administering microcarriers to the area of defect involves sphere packing. In some examples, the microcarriers administered to the bone defect are substantially equal in size, and the packing density is about 10 to about 60%, or about 20% to about 50%, or about 30% to about 40%, or at about 15, 25, 35, 45, or 55%. In some other examples, the microcarriers administered to the bone defect are not equal in size, and the packing density is about 10 to about 90%, or about 20% to about 80%, or about 30% to about 70%, or about 40% to about 60%, or at about 15, 25, 35, 45, 50, 55, 65, 75 or 85%.

In some examples, administering the implantable construct or the one or more cell-free polycaprolactone (PCL) microcarriers result in the secretion of paracrine factors that promote proliferation and/or migration, and/or inhibit apoptosis of endogenous chondrocytes and/or osteoblasts, resulting in increased therapeutic efficacy as compared to other tissue formation/tissue regeneration methods currently available. Examples of paracrine factors include but are not limited to fibroblast growth factors, Hedgehog proteins, Wnt proteins, TGF-β family proteins, epidermal growth factor, cytokines, and the interleukins.

In some examples, the mesenchymal stromal cells used in the method and implantable construct of the present invention can be replaced by other types of stem cells as mentioned herein, including but not limited to topipotent stem cells, pluripotent stem cells including induced pluripotent stem cells, multipotent stem cells, and embryonic stem cells.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXAMPLES

The following examples illustrate methods by which aspects of the invention may be practiced or materials suitable for practice of certain embodiments of the invention may be prepared.

### Example 1—Evaluation of Critical Parameters to Achieve Efficient Chondrogenic Differentiation of heMSC-LPCL Microcarrier Constructs

Schematic of experimental design—Stage 1: heMSCs attached to LPCL microcarriers were seeded as chondrogenic heMSC-microcarrier constructs at either day 3 (early log phase with 21% cell confluency), day 5 (mid log phase with 53% cell confluency) or day 7 (late log phase with 73% cell confluency), using different cell numbers per construct. The critical cell confluency and cell number per construct were then identified by evaluating cell growth and differentiation output at day 21. Stage 2: heMSC-microcarrier constructs generated under critically-defined conditions as identified at Stage 1 were evaluated for the effect of compaction. heMSCs were seeded (i) with or without centrifugation and (ii) with or without agitation. The effects of centrifugation and/or agitation were determined by evaluating cell growth and differentiation output at day 21.

Results

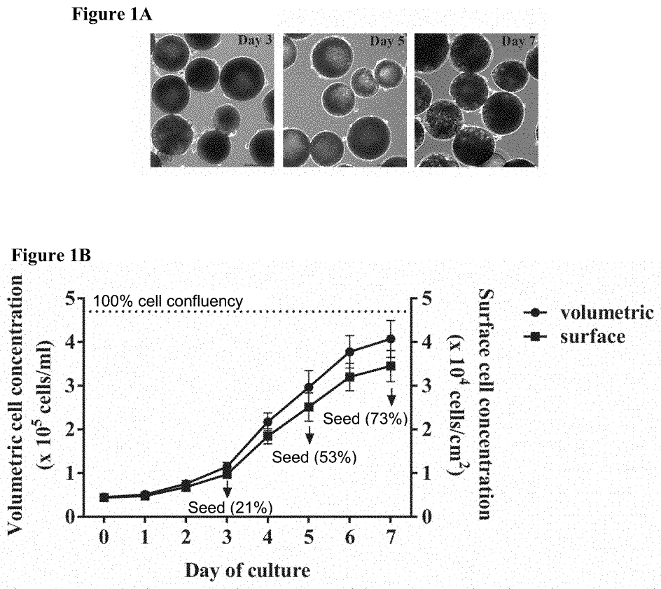

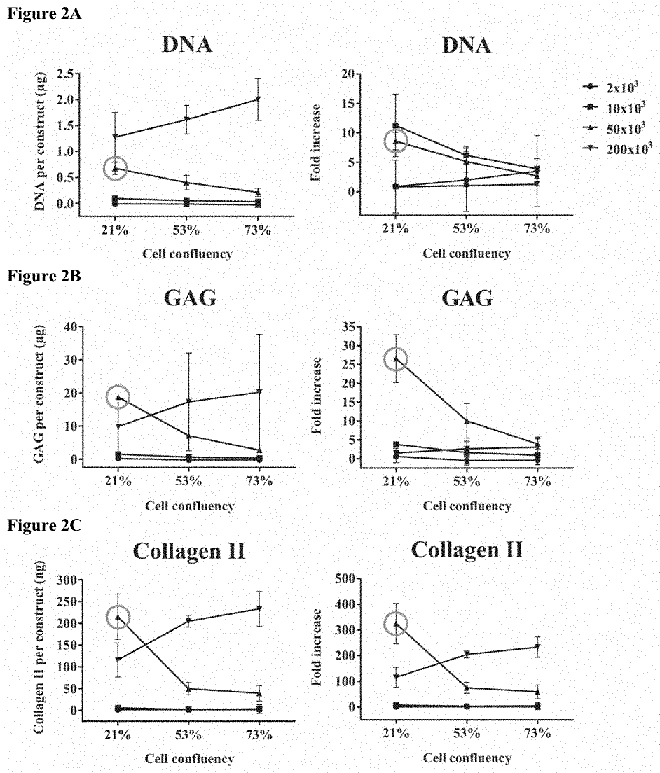

Cell confluency and cell numbers per construct are important parameters required to create critically-defined hMSC-LPCL MC constructs. Specifically, seeding 50×10³ cells at 21% cell confluency per hMSC-LPCL construct (i.e. at day 0 of differentiation) resulted in most efficient cell growth (DNA content of 0.675±0.166 pg per construct and fold increase of 8.59±1.48 by day 21 of differentiation) and chondrogenic differentiation (GAG content of 18.8±4.46 pg per construct and fold increase of 26.6±6.31 by 21 days of differentiation); Collagen II content of 215±52.0 ng per construct and fold increase of 325±78.5 by 21 days of differentiation) (FIGS. 1, 2 and Table 1).

TABLE 1
Characteristics of critically-defined hMSC-LPCL MC constructs (end product/off the shelf
product that can be used directly for transplantation). Day refers to day of differentiation
of seeded hMSC-LPCL MC construct. Day 14 through Day 28 chondrogenically differentiated
hMSC-LPCL MC constructs can be used for transplantations. For the rabbit animal study,
day 21 chondrogenically differentiated hMSC-LPCL MC constructs were used, which is a
common and recommended stage of differentiation for hMSC cells-only.

| | DNA | GAG | GAG/DNA | Collagen II | Collagen II/DNA |
|---|---|---|---|---|---|
| | (μg) | (μg) | (μg/μg) | (ng) | (ng/μg) |
| Day 14 | 0.372 ± 0.059 | 8.806 ± 2.530 | 23.531 ± 4.127 | 82.629 ± 31.436 | 224.891 ± 121.265 |
| Day 21 | 0.441 ± 0.091 | 15.015 ± 5.048 | 24.833 ± 7.429 | 20.961 ± 12.417 | 32.439 ± 22.990 |
| | 0.660 ± 0.103 | 16.000 ± 3.249 | 27.578 ± 2.090 | 131.953 ± 76.863 | 301.400 ± 163.184 |
| | 0.675 ± 0.116 | 18.759 ± 4.459 | 33.336 ± 5.318 | 215.125 ± 52.028 | 323.655 ± 79.932 |
| Day 28 | 0.875 ± 0.162 | 48.961 ± 9.714 | 55.869 ± 1.960 | 603.059 ± 156.093 | 683.059 ± 242.661 |

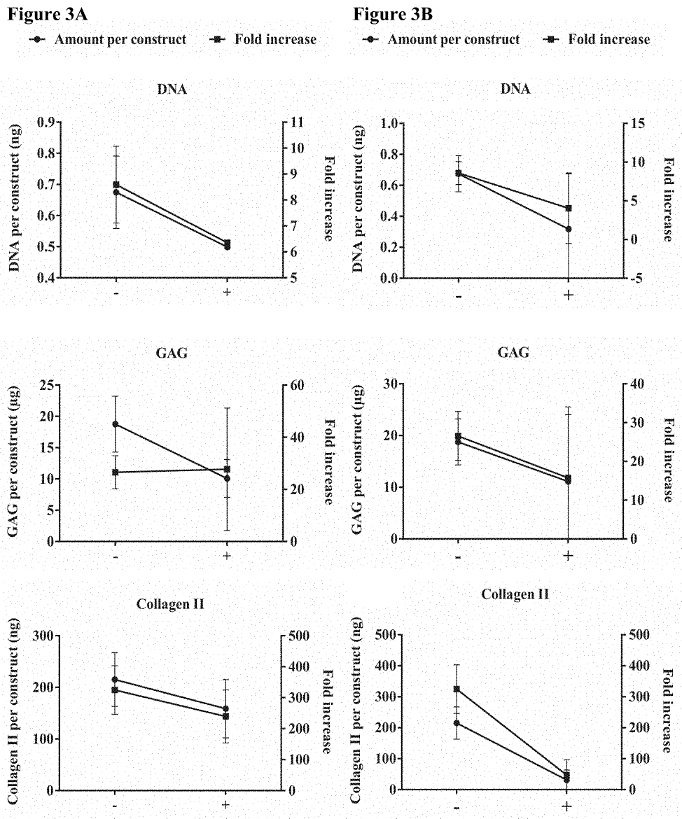

Construct compaction by applying centrifugation at seeding or continuous agitation throughout differentiation are not important to create critically-defined hMSC-LPCL MC constructs. Specifically, these parameters were shown to attenuate cell growth and reduce chondrogenic output (FIG. 3). In fact, culturing hMSC attached on LPCL MC without centrifugation at seeding (i.e. day 0 of differentiation), and without continuous agitation throughout the course of chondrogenic differentiation yielded the best results.

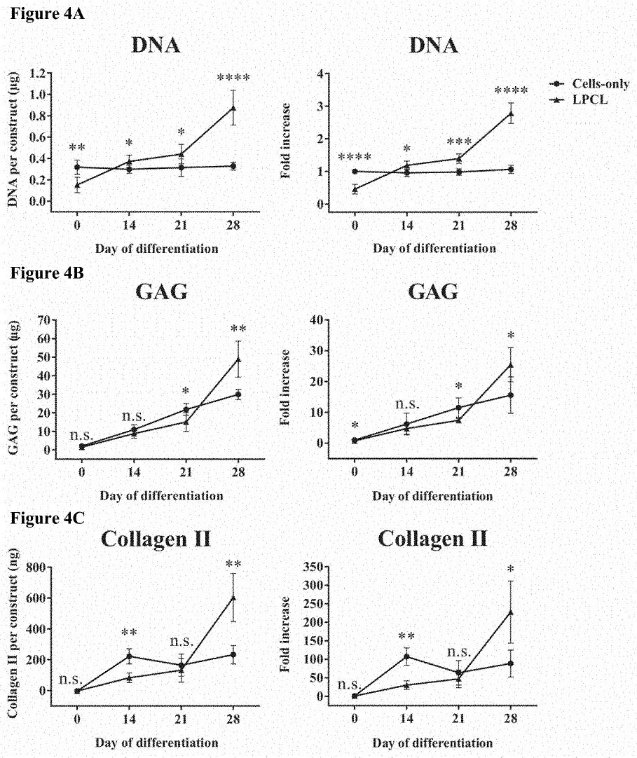

Critically-defined hMSC-LPCL MC constructs increased cellular proliferation in terms of DNA content (2.62 fold) over their equivalent cells-only counterparts (DNA content of 0.875±0.162 pg per hMSC-LPCL MC construct as opposed to 0.329±0.037 pg per cell-only pellet; DNA fold increase of 2.78±0.315 per hMSC-LPCL MC construct as opposed to 1.06±0.125 per cell-only pellet by day 28 of differentiation). Critically-defined hMSC-LPCL MC constructs improved chondrogenic output in terms of proteoglycan (1.63 fold) and Collagen II (2.57 fold) content over their equivalent cells-only counterparts (GAG content of 49.0±9.71 pg per hMSC-LPCL MC construct as opposed to 29.9±2.73 pg per cell-only pellet; GAG fold increase of 25.5±5.50 per hMSC-LPCL MC construct as opposed to 15.6±5.90 per cell-only pellet by day 28 of differentiation) (Collagen II content of 603±156 ng per hMSC-LPCL MC construct as opposed to 232±59.3 ng per cell-only pellet; Collagen II fold increase of 228±84.0 per hMSC-LPCL MC construct as opposed to 88.8±36.4 per cell-only pellet by day 28 of differentiation). (see FIG. 4).

In Vivo Transplantation

TABLE 2
Experimental groups tested for in vivo transplantation of heMSC-
LPCL microcarrier constructs in a rabbit endochondral defect model.

| Groups | |
|---|---|
| 1 | Defect only |
| 2 | Defect + chondrogenically differentiated hMSC only |
| | (without MC) |
| 3 | Defect + LPCL MC only (without cells) |
| 4 | Defect + undifferentiated hMSC-LPCL construct |
| 5 | Defect + chondrogenically differentiated hMSC-LPCL |
| | construct |
| 6 | No defect |

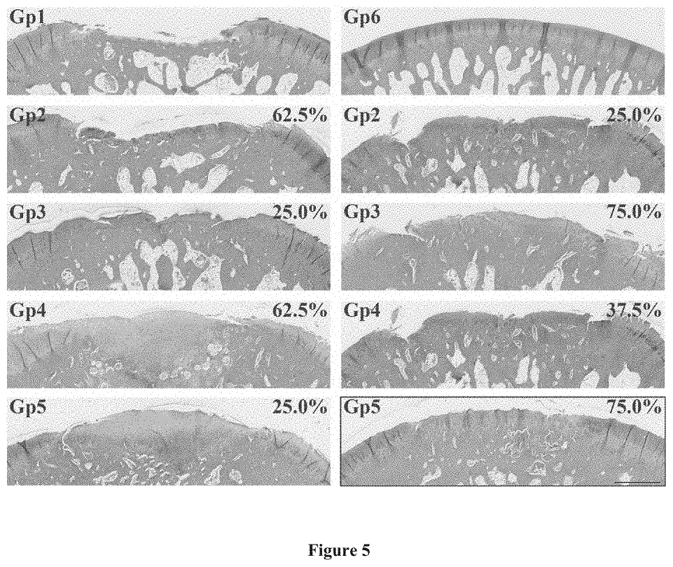

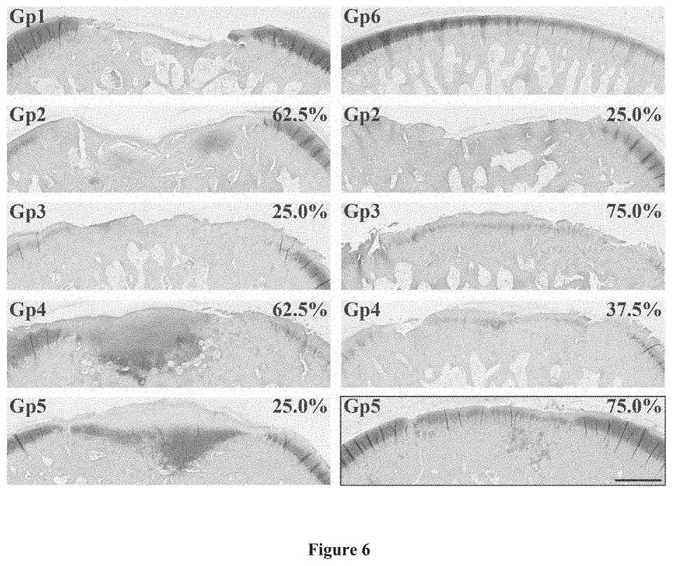

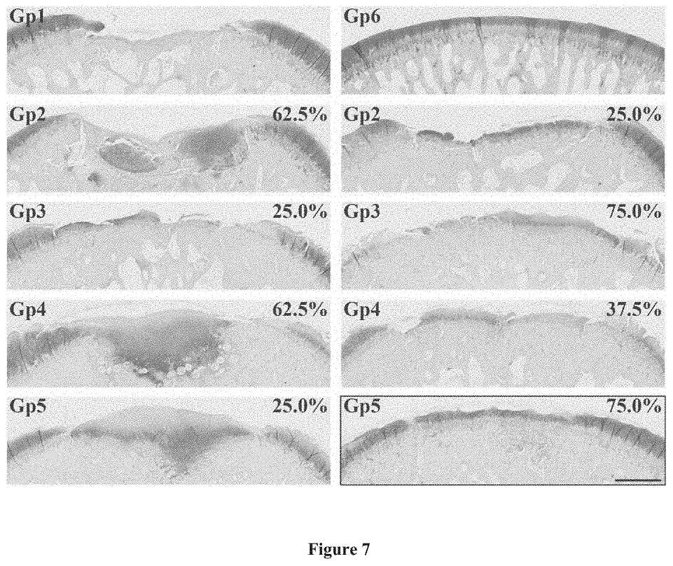

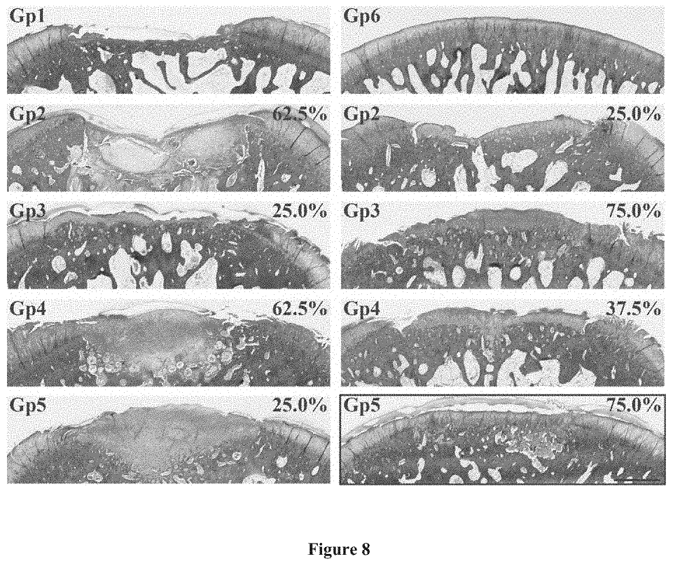

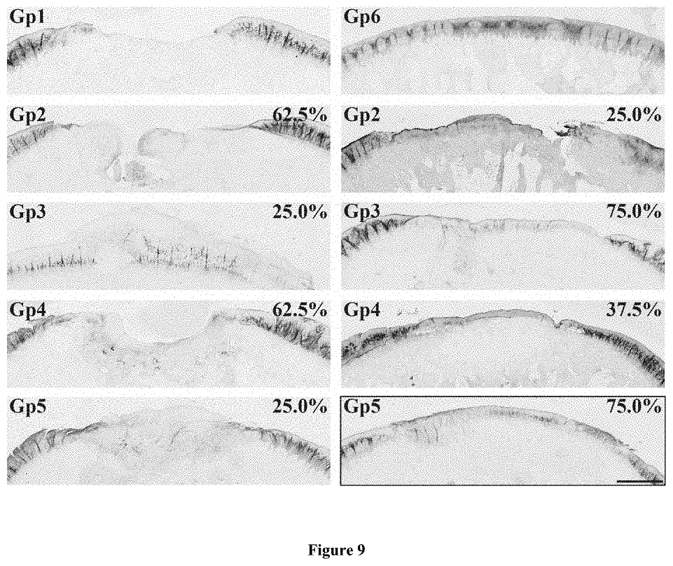

Transplantation of chondrogenically differentiated critically-defined hMSC-LPCL MC constructs in rabbit cartilage lesions results in the best cartilage generation and healing/repair outcomes in terms of general tissue morphology (as shown by H&E staining in FIG. 5), proteoglycan (as shown by Safranin O and Alcian Blue staining in FIGS. 6 and 7) and Collagen II content (as shown by Masson's Trichrome staining and Collagen II immunostaining in FIGS. 8 and 9) at 5 months post-transplantation. This is done in comparison to 5 other animal groups including those (i) with lesion only and without any implants, (ii) with lesion and only chondrogenically differentiated hMSC without any LPCL MC implanted, (iii) with lesion and only empty LPCL MC without any cells implanted, (iv) with lesion and undifferentiated critically-defined hMSC-LPCL MC constructs implanted, and (v) without any lesions (wild type) (see Table 2). Based on qualitative histological stainings, transplantation of chondrogenically differentiated critically-defined hMSC-LPCL MC constructs (Gp5) yielded the best results, followed by empty LPCL MC without any cells implanted (Gp3), followed by undifferentiated critically-defined hMSC-LPCL MC constructs implanted (Gp4) and lastly only chondrogenically differentiated hMSC without any LPCL MC implanted (Gp2). Specifically, transplantation of chondrogenically differentiated critically-defined hMSC-LPCL MC constructs (Gp5) resulted in the best cartilage generation, as evident from the intensity of the above-mentioned stainings, as well as tissue healing, as evident from the filling of the lesion, surface regularity of neo-formed tissue in the lesion and the bonding of neo-formed tissue with adjacent native cartilage. (highlighted in black boxes in FIGS. 5 through 9). More importantly, it resulted in not only the lowest number of transplant cases (25.0%) displaying poor healing outcomes, which are more similar to animals with lesions only and without any implants, as shown in the left columns of FIGS. 5 through 9, but also crucially, it resulted in the highest number of transplant cases (75.0%, black boxes) displaying good healing outcomes, which are more similar to animals without any lesions, as shown in the right columns of FIGS. 5 through 9.

Transplantation of chondrogenically differentiated critically-defined hMSC-LPCL MC constructs in rabbit cartilage lesions also results in the best cartilage generation and healing/repair outcomes in terms of histological scoring for microscopic cartilage healing evaluation at 5 months post-transplantation. This is done in comparison to 5 other animal groups including those (i) with lesion only and without any implants, (ii) with lesion and only chondrogenically differentiated hMSC without any LPCL MC implanted, (iii) with lesion and only empty LPCL MC without any cells implanted, (iv) with lesion and undifferentiated critically-defined hMSC-LPCL MC constructs implanted, and (v) without any lesions (wild type). Based on quantitative histological scoring (O'Driscoll scoring), transplantation of chondrogenically differentiated critically-defined hMSC-LPCL MC constructs (Gp5) yielded the best results, followed by empty LPCL MC without any cells implanted (Gp3), followed by undifferentiated critically-defined hMSC-LPCL MC constructs implanted (Gp4) and lastly only chondrogenically differentiated hMSC without any LPCL MC implanted (Gp2). Specifically, transplantation of chondrogenically differentiated critically-defined hMSC-LPCL MC constructs (Gp5) achieved (i) the highest mean scores for 8 out 12 categories, (ii) the greatest total sums, (iii) statistically most similar to animals with no defect in 4 out of 12 categories. (see Tables 3 to 5)

Importantly, the results show that it is the critically-defined combination of stem cell type/status attached on LPCL MC that enables it to have the best cartilage generation and healing abilities in vivo. This is supported by the results where transplantation of either empty LPCL MC without any cells or undifferentiated critically-defined hMSC-LPCL construct results in the second-best and third-best cartilage generation and healing/repair outcomes respectively, in terms of general tissue morphology (as shown by H&E staining in FIG. 5), proteoglycan content (as shown by Safranin O and Alcian Blue staining in FIGS. 6 and 7), Collagen II content (as shown by Masson's Trichrome staining and Collagen II immunostaining in FIGS. 8 and 9) and histological scoring for microscopic cartilage healing evaluation at 5 months post-transplantation. Specifically, transplantation of empty LPCL MC (Gp3) outperformed implantation of undifferentiated critically-defined hMSC-LPCL MC constructs (Gp4) and further outperformed chondrogenically differentiated hMSC without any LPCL MC implanted (Gp2). This demonstrates not only the importance of LPCL MC itself in orchestrating the cartilage generation and healing outcomes, but it shows that the type of cell attached on the LPCL MC (i.e. chondrogenically differentiated stem cells are better than undifferentiated stem cells) is critical in determining the efficacy of cartilage generation and healing in vivo. The results showed that transplantation of cells-only (Gp2) yielded the poorest cartilage generation and healing outcomes in rabbit cartilage lesions, suggesting that the chondrogenically differentiated critically-defined hMSC-LPCL MC constructs as disclosed herein can be used as an effective allogeneic stem cell therapeutic product for the healing of cartilage-related disorders. (see Tables 2 to 5 and FIGS. 5 to 9)

Scoring criteria for microscopic cartilage healing evaluation

TABLE 3
Scoring criteria for microscopic cartilage healing
of rabbit knee joints from respective experimental
groups at 5 months after transplantation.

Score

(A) Overall defect evaluation (throughout the entire defect depth)

| | | |
|---|---|---|
| 1. | Percent filling with neo-formed tissue | |
| | 100% | 3 |
| | >50% | 2 |
| | <50% | 1 |
| | 0% | 0 |

(B) Cartilage evaluation (within upper 1mm of the defect)

| | | |
|---|---|---|
| 2. | General morphology of neo-formed tissue | |
| | Exclusively hyaline cartilage | 4 |
| | Mainly hyaline cartilage | 3 |
| | Fibrocartilage (spherical morphology observed | 2 |
| | with ≥75% of cells) | |
| | Only fibrous tissue or bone (spherical morphology | 1 |
| | observed with <75% of cells) | |
| | No tissue | 0 |
| 3. | Thickness of neo-formed tissue | |
| | Similar to the surrounding cartilage (75% to | 3 |
| | 100% of adjacent native cartilage) | |
| | Greater than surrounding cartilage (>100% of | 2 |
| | adjacent native cartilage) | |
| | Less than the surrounding cartilage (<75% of | 1 |
| | adjacent native cartilage) | |
| | No cartilage (0% of adjacent native cartilage) | 0 |
| 4. | Joint surface regularity of neo-formed tissue | |
| | Smooth, intact surface | 3 |
| | Surface fissures (<25% of neo-surface thickness) | 2 |
| | Deep fissures (≥25% of neo-surface thickness) | 1 |
| | Complete disruption of the neo-surface | 0 |
| 5. | Structural integrity of neo-formed tissue | |
| | Normal | 3 |
| | Slight disruption, including cysts | 2 |
| | Moderate disruption | 1 |
| | Severe disintegration | 0 |
| 6. | Extent of neo-tissue bonding with adjacent cartilage | |
| | Complete on both edges | 3 |
| | Complete on one edge | 2 |
| | Partial on both edges | 1 |
| | Without continuity on either edge | 0 |
| 7. | GAG content (Safranin O/Alcian Blue staining) | |
| | within neo-tissue | |
| | Normal (75%-100% of adjacent native cartilage) | 3 |
| | Moderately reduced (50-75% of adjacent native cartilage) | 2 |
| | Severely reduced (25-50% of adjacent native cartilage) | 1 |
| | Absent or no cartilage (0-25% of adjacent native cartilage) | 0 |
| 8. | Collagen content (Masson's Trichrome/Collagen | |
| | II staining) within neo-tissue | |
| | Normal (75%-100% of adjacent native cartilage) | 3 |
| | Moderately reduced (50-75% of adjacent native cartilage) | 2 |
| | Severely reduced (25-50% of adjacent native cartilage) | 1 |
| | Absent or no cartilage (0-25% of adjacent native cartilage) | 0 |
| 9. | Cellularity within neo-formed tissue | |
| | Normal | 3 |
| | Slight hypo-cellularity | 2 |
| | Moderate hypo-cellularity | 1 |
| | Severe hypo-cellularity or no cells | 0 |
| 10. | Chondrocyte clustering within neo-formed tissue | |
| | None at all | 3 |
| | <25% chondrocytes | 2 |
| | ≥25% chondrocytes | 1 |
| | No chondrocytes present (no cartilage) | 0 |
| 11. | Cellularity and GAG content of adjacent cartilage | |
| | Normal cellularity with normal Safranin O staining | 3 |
| | Normal cellularity with moderate Safranin O staining | 2 |
| | Clearly less cells with poor Safranin O staining | 1 |
| | Few cells with no or little Safranin O staining or no | 0 |
| | cartilage | |

(C) Subchondral bone evaluation (within bottom 2 mm of defect)

| | | |
|---|---|---|
| 12. | Subchondral bone morphology | |
| | Normal, trabecular bone | 4 |
| | Trabecular, with some compact bone | 3 |
| | Compact bone | 2 |
| | Compact bone and fibrous tissue | 1 |
| | Only fibrous tissue or no tissue | 0 |

| | |
|---|---|
| Maximum score | 38 |

Histological Scores for Microscopic Cartilage Healing Evaluation

TABLE 4
Summary of histological scores for microscopic cartilage healing of
rabbit knee joints from respective experimental groups at 5 months
after transplantation. Bold numbers represent the highest mean score(s)
achieved. Implantation of chondrogenically differentiated heMSC-LPCL
microcarrier constructs (Group 5) achieved the best cartilage healing
outcomes, as it scored the highest mean for 8 out of 12 categories
and the greatest total sums, as compared to group 1 through 4.

| Groups (n = 8) | | | | | |
|---|---|---|---|---|---|
| 1 | 2 | 3 | 4 | 5 | 6 |

(A) Overall defect evaluation

| | | | | | | | |
|---|---|---|---|---|---|---|---|
| 1. | Percent filling with | 1.63 ± | 1.75 ± | 2.00 ± | 2.00 ± | 2.43 ± | 3.00 ± |
| | neo-formed tissue | 1.06 | 1.04 | 0.58 | 1.31 | 0.79 | 0.00 |

(B) Cartilage evaluation

| | | | | | | | |
|---|---|---|---|---|---|---|---|
| 2. | General morphology of | 1.88 ± | 2.13 ± | 2.48 ± | 2.13 ± | 2.57 ± | 4.00 ± |
| | neo-formed tissue | 1.36 | 0.64 | 0.69 | 1.46 | 1.13 | 0.00 |
| 3. | Thickness of neo-formed | 1.50 ± | 1.38 ± | 2.14 ± | 1.88 ± | 2.14 ± | 3.00 ± |
| | tissue | 1.31 | 1.06 | 1.07 | 1.36 | 1.07 | 0.00 |
| 4. | Joint surface regularity of | 1.00 ± | 1.13 ± | 1.43 ± | 1.13 ± | 2.29 ± | 3.00 ± |
| | neo-formed tissue | 0.76 | 0.99 | 0.53 | 0.99 | 0.49 | 0.00 |
| 5. | Structural integrity of | 1.00 ± | 1.13 ± | 1.29 ± | 1.25 ± | 1.71 ± | 3.00 ± |
| | neo-formed tissue | 0.93 | 0.83 | 0.76 | 1.04 | 0.76 | 0.00 |
| 6. | Extent of neo-tissue | 1.25 ± | 1.38 ± | 2.25 ± | 1.38 ± | 2.14 ± | 3.00 ± |
| | bonding with adjacent | 1.17 | 1.06 | 0.71 | 1.30 | 0.90 | 0.00 |
| | cartilage | | | | | | |
| 7. | GAG content within neo- | 1.38 ± | 1.38 ± | 1.57 ± | 1.38 ± | 1.29 ± | 3.00 ± |
| | tissue | 1.41 | 1.19 | 1.27 | 1.06 | 0.95 | 0.00 |
| 8. | Collagen content within | 1.63 ± | 1.88 ± | 2.00 ± | 1.88 ± | 2.00 ± | 3.00 ± |
| | neo-tissue | 1.30 | 1.36 | 1.16 | 1.25 | 1.29 | 0.00 |
| 9. | Cellularity within neo- | 1.50 ± | 1.00 ± | 1.14 ± | 1.13 ± | 1.00 ± | 3.00 ± |
| | formed tissue | 1.31 | 1.07 | 0.90 | 0.83 | 0.82 | 0.00 |
| 10. | Chondrocyte clustering | 1.00 ± | 0.88 ± | 1.00 ± | 1.25 ± | 1.14 ± | 3.00 ± |
| | within neo-formed tissue | 0.93 | 0.64 | 0.82 | 0.89 | 0.90 | 0.00 |
| 11. | Cellularity and GAG | 2.88 ± | 2.38 ± | 2.86 ± | 3.00 ± | 3.00 ± | 3.00 ± |
| | content of adjacent | 0.35 | 1.06 | 0.38 | 0.00 | 0.00 | 0.00 |
| | cartilage | | | | | | |

(C) Subchondral bone evaluation

| | | | | | | | |
|---|---|---|---|---|---|---|---|
| 12. | Subchondral bone | 3.75 ± | 2.88 ± | 4.00 ± | 3.63 ± | 4.00 ± | 4.00 ± |
| | morphology | 0.71 | 1.55 | 0.00 | 1.06 | 0.00 | 0.00 |

| | | | | | | |
|---|---|---|---|---|---|---|
| Total sum (A&B max = 34) | 16.6 ± | 16.4 ± | 20.1 ± | 18.4 ± | 21.7 ± | 34.0 ± |
| | 9.53 | 6.78 | 6.44 | 10.1 | 7.30 | 0.00 |
| Total sum (A-C max = 38) | 20.4 ± | 19.3 ± | 24.1 ± | 22.0 ± | 25.7 ± | 38.0 ± |
| | 9.96 | 7.32 | 6.44 | 10.1 | 7.30 | 0.00 |

Statistical Analysis by Comparing to Positive Control Group 6

TABLE 5
Statistical analysis of histological scores for microscopic cartilage healing of rabbit knee joints from respective
experimental groups to that of positive control Group 6 (wild type). Group 1 (defect only) was significantly
different from Group 6 (no defect) in 6 out of 12 categories, which showed that our experimental model yielded
significantly different results and could provide a basis for comparing the other groups. Group 5 was not
significantly different from Group 6 in 6 areas (highlighted in grey) while other groups were, suggesting
that Group 5's healing outcomes were the best as they scored the closest to that of Group 6.

| | | Experimental group | | | | |
|---|---|---|---|---|---|---|
| | | 1 | 2 | 3 | 4 | 5 |
| Histological | 1. Percent filling with neo-formed tissue | ns | ns | ns | ns | ns |
| Scoring | 2. General morphology of neo-formed tissue | * | * | ns | * | ns |
| | 3. Thickness of neo-formed tissue | ns | ns | ns | ns | ns |
| | 4. Joint surface regularity of neo-formed tissue | *** | *** | * | *** | ns |
| | 5. Structural integrity of neo-formed tissue | ** | ** | * | * | ns |
| | 6. Extent of neo-tissue bonding with adjacent cartilage | * | * | ns | * | ns |
| | 7. GAG content within neo-tissue | ns | ns | ns | ns | ns |
| | 8. Collagen content within neo-tissue | ns | ns | ns | ns | ns |
| | 9. Cellularity within neo-formed tissue | * | * | * | * | * |
| | 10. Chondrocyte clustering within neo-formed tissue | *** | *** | ** | ** | ** |
| | 11. Cellularity and GAG content of adjacent cartilage | ns | ns | ns | ns | ns |
| | 12. Subchondral bone morphology | ns | ns | ns | ns | ns |
| | Average sum without bone | ** | ** | * | * | ns |
| | Average sum with bone | ** | ** | * | * | ns |
| | Microcarrier remnants | ns | ns | ** | ns | ns |
| | Cartilage thickness in defect | na | | | | |
| | Cartilage thickness in adjacent native cartilage | | | | | |
| | Ratio of cartilage thickness in defect over adjacent native cartilage | | | | | |

p values, na = not applicable, ns = p > 0.05, ^{#}ns = p > 0.03, * p < 0.03, ** p < 0.001 and *** p < 0.0001.

Transplantation of stem cell-covered LPCL MC also resulted in lesser MC remnants (at least 2.39 fold less) in the cartilage lesions, as compared to that of empty LPCL MC at 5 months post-transplantation. Specifically, transplantation of chondrogenically differentiated or undifferentiated critically-defined hMSC-LPCL MC constructs resulted in only 10.3±12.1 and 10.1±10.9 MC remnants respectively while transplantation of empty LPCL MC without cells results in 24.6±17.2 MC remnants at 5 months post-transplantation (Table 6). This is likely due to enhanced enzymatic degradation of LPCL by the stem cells including hMSC (undifferentiated or chondrogenically differentiated). This further supports the importance of using critically-defined combination of stem cell attached on LPCL MC as a combination therapeutic product to achieve healing of cartilage lesions. It is also observed that the microcarrier remnants, regardless of whether they were initially stem cell covered or not, are absent from the cartilage layer but are primarily found in the bone layer, which could contribute to better cartilage healing outcomes, as observed (Table 6).

Quantification of Microcarrier Remnants

TABLE 6
Quantification of microcarrier remnant numbers. Transplantation
of chondrogenically differentiated heMSC-LPCL microcarrier constructs
(Group 5) was better than that of empty LPCL microcarriers without
any cells (Group 3) as it resulted in 2.39 fold fewer microcarrier
remnants at 5 months after transplantation.

| | Groups (n = 8) | | | | | |
|---|---|---|---|---|---|---|
| | 1 | 2 | 3 | 4 | 5 | 6 |
| (A) | 0.00 ± | 0.00 ± | 24.6 ± | 10.1 ± | 10.3 ± | 0.0 0.00 |
| Microcarrier | 0.00 | 0.00 | 17.2 | 10.9 | 12.1 | |
| remnants | | | | | | |

### Example 2—hMSC-Covered LPCL MC Constructs for Allogenic Bone Regeneration

This example describes the development of a combined stem cell-biomaterial therapeutic product, which is scalable and bioimplantable for allogenic bone regeneration, in the form of critically-defined 50% hMSC-covered LPCL MC constructs.

Materials and Methods

PCL (average Mn 45 kDa, Cat. No. 704105) and poly-L-lysine hydrobromide (PLL) (MW 70-150 kDa, Cat. No. P6282) were sourced from Sigma-Aldrich. Fibronectin were purchased from Biological Industries. All chemical reagents were obtained from Sigma-Aldrich and all culture media and supplements were bought from ThermoFisher Scientific.

Fabrication of PCL MC

Porous PCL MC were fabricated using a two phase flow microfluidic device as previously reported. Briefly, PCL droplets were collected in a glass cylinder containing 70%-95% ethanol, soaking in ethanol leads to solidification of PCL droplets into porous PCL MC (with low density of 1.06 g/L and diameter, 162±9 μm). The MCs were then incubated in 5 mol/L sodium hydroxide (NaOH) for an hour to enhance the surface property for extracellular matrix (ECM) coating.

For better cell adhesion and spreading, MCs were coated with 3 layers of ECM—2 μg/cm² of FN, 1 μg/cm² of PLL and 2 μg/cm² of FN, at room temperature. Coated MCs were washed with phosphate-buffered saline (PBS) and stored at 4° C. before use. The coated porous PCL MCs is designated as LPCL.

Ethics of Obtaining Human Early MSC (heMSC)

heMSC were supplied by Jerry Chan from the National University of Singapore. Fetal tissues were obtained from 13 week old, clinically terminated pregnancies with the approval by the Domain Specific Review Board of National University Hospital, Singapore (DREB-D-06-154). heMSC were isolated from fetal bone marrow by plastic adherence and characterized using methods known in the art.

Cell Culture and Media

Cells were cultured in αMEM medium supplemented with 10% (vol/vol) fetal bovine serum (FBS, ThermoFisher Scientific) with 50 U/mL penicillin and 50 mg/mL streptomycin (ThermoFisher Scientific) and maintained in CO₂ humidified incubator at 37° C. Single cell suspension of heMSCs was prepared by trypsinization as previously described. heMSC at passage 6-10 were used for all experiments described here.

Cultivation of heMSC on LPCL in Spinner Flask

Seeding of heMSC in spinner flasks were previously described. Briefly, heMSCs (4.5×10⁴ cell/mL) were harvested by trypsinization and inoculated onto 700 mg of LPCL in 125 mL plastic spinner flasks containing 50 mL of α10 culture medium. The culture was left static for 2 hours followed by continuous stirring at 40 rpm with 50% medium changed every 2 days for 6 days.

Immunophenotypic Analysis

Live cells harvested from spinner cultures were analyzed with CD34 (1:10), CD70 (1:10), CD90 (1:10) and CD105 (1:20) (source from Bio-legend) following protocols described previously.

Multiplex Cytokine Analysis

Cytokines were measured using Luminex® human cytokine multiplex kit (Thermofisher Scientific). Calibration curves from recombinant cytokine standards were prepared with serial dilutions in the same media as the culture supernatants (α10). High and low reference points were included to determine cytokine recovery. Standards and reference points were measured in triplicate, each sample was measured once, and blank values were subtracted from all reading. All assays were carried out directly in a 96-well filtration plate (Millipore) at room temperature and protected from light. Briefly, wells were pre-wetted with 1004 μL PBS containing 1% bovine serum albumin (BSA), then beads (5000 beads per cytokine) together with a either a standard, sample, reference point, or blank were added in a final volume of 100 μL, and incubated together at room temperature for 30 minutes with continuous shaking. Beads were washed three times with 100 μL PBS containing 1% BSA and 0.05% Tween-20. A cocktail of biotinylated antibodies (50 μl/well) was added to the beads for 30 minutes incubation with continuous shaking. Beads were again washed three times, and then streptavidin-phycoerythrin was added for 10 minutes. Beads were again washed three times and resuspended in 125 μL of PBS containing 1% BSA and 0.05% Tween-20. The fluorescence intensity of the beads was measured using the Bio-Plex array reader (Bio-Rad). Bio-Plex manger software with five-parametric-curve fitting was used for data analysis.

In Vitro 2D Osteogenic Differentiation of heMSC on LPCL Microcarriers

6-well culture plates were coated for 1 hour at 37° C. with 0.01% rat tail collagen I (BD Biosciences). Then, cells were seeded with density of 2×10⁴ cells/cm² containing osteogenic differentiation medium: Dulbecco's modified Eagle's medium (DMEM) supplemented with 10% fetal bovine serum (FBS), 10 mM β-glycerophosphate, 10 nM dexamethasone and 0.2 mM ascorbic acid. The cultures were incubated for 21 days with medium changed every other day.

Calcium Deposition Assay

The osteogenic differentiated cells cultured on 6-well culture plate were washed three times with PBS (Mg²⁺, Ca²⁺ free) and then incubated with 0.5N acetic acid for 60 minutes at room temperature. Eluted calcium was quantified using a calcium assay kit (BioAssay System) according to manufacturer's instructions. Results were normalized to total cell count measured by nuclei counting (Nucleocounter, ChemoMetec).

In Vivo Bone Formation

heMSCs were cultured on LPCL microcarriers or tissue culture plastic monolayers (MNL). Cells were expanded to reach 50% confluence on LPCL (3 days) and 100% confluence on LPCL (6 days) in spinner flask. For MNL cultures, cells were expanded to reach about 80% confluence before used.

Implants for transplantation were prepared by mixing 100 μl fibrin glue (Tisseel Kit, Baxter) with 30 mg of hydroxyapatite powder together with the following conditions on a 96-well plate:
1) Fibrin gel and HA (Empty control)
2) Free-cell LPCL (LPCL only) (contain about 960 microcarriers)
3) MSCs harvested from MNL cultures (MNL MSCs)
4) 50% heMSCs-covered LPCL (50% MSCs LPCL) (contain about 960 microcarriers)
5) 100% heMSCs-covered LPCL (100% MSCs LPCL) (contain about 500 microcarriers)

4×10⁴ cells were added to the implants where cells were involved. After fibrin glue had polymerized, each implants was incubated in growth media for 2 to 3 hours until the surgery.

Calvarial Defect Surgery

All animal experiments were performed with IACUC approval with institutional guidelines (Biological Resource Center, IACUC #130878 and #171239). The calvarial bone defect protocol as previously described was used. Briefly, two 5 mm defects were created on NIH nude male rats (11-12 weeks, 280-305 g), anesthetized under isoflurane. Implants were gently washed in PBS and placed into the defects. The incision was closed with 7.0 VICRYL absorbable sutures (BD) and Vetbond™ Tissue adhesive (3M). Mice were administered 10 mg/kg antibiotic Baytril (Sigma-Aldrich) and 0.05 mg/kg analgesics Buprenophine (Sigma-Aldrich) for 3 days.

TABLE 7
Experimental groups tested for in vivo transplantation of heMSC-
LPCL microcarrier constructs in a mouse calvarial defect model.

| Groups | |
|---|---|
| 1 | Defect + fibrin gel and HA (nominally empty control) |
| 2 | Defect + cell-free LPCL (LPCL only) |
| 3 | Defect + MSCs harvested from MNL cultures (MNL MSCs) |
| 4 | Defect + 50% heMSCs-covered LPCL (50% MSCs LPCL) |
| 5 | Defect + 100% heMSCs-covered LPCL (100% MSCs LPCL) |
| 6 | Autograft |

Ex Vivo Micro-CT Analysis

Calvaria defects were imaged at 16 weeks to evaluate new bone formation at the defect site using micro-CT (Bruker). They were scanned using 0.8 degree angle rotation step size, 35 resolution, 1.0 Al filter, 100 kV, and 100 μA. Reconstruction was done using the manufacturer's software (Dataviewer, NRecon and CTAn), with beam hardening at 30%, smoothing at 3 and ring artifact at 5. In order to ensure only new bone formation was measured, quantification of bone volume was performed by evaluating in the central 4 mm of the defect.

Histology

Bone samples were harvested 16 weeks after transplantation. Samples were fixed in 10% neutral-buffered saline (Sigma-Aldrich), decalcified and embedded in paraffin using Histo-Clear (National Diagnostics). Sections (5 μm) were deplasticized and stained with hematoxylin and eosin (H&E; Sigma-Aldrich) and Masson's trichrome stains (Sigma-Aldrich).

To estimate the number of microcarriers left in the implants, for each sample containing LPCL MC, the number of microcarriers of 6 sections were counted by using Image J software.

Results

Cell Growth Kinetics

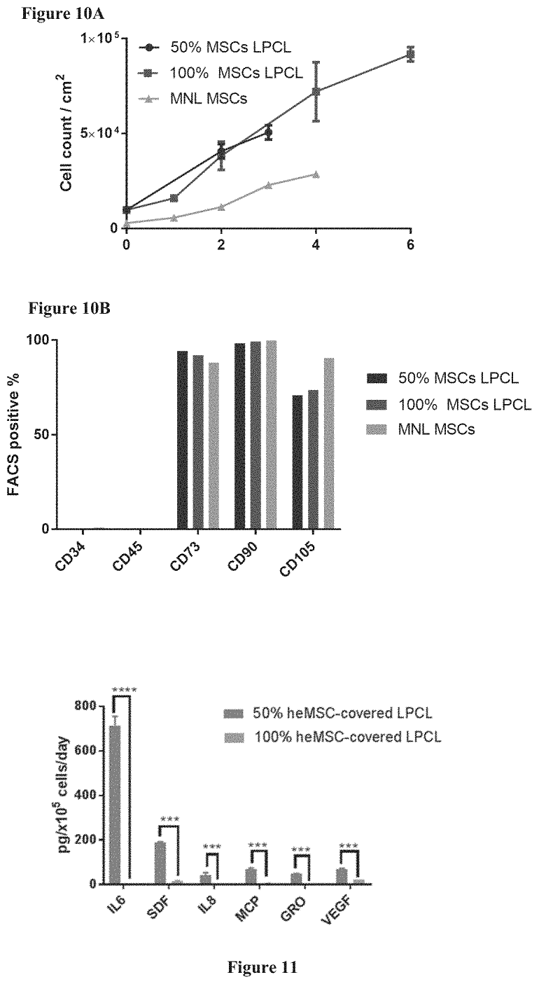

heMSCs were cultured to 50% (Day 3) and 100% (Day 6) confluence on LPCL MCs in spinner flask cultures under agitation (40 rpm). FIG. 10A show the highest cell density on day 6, when cells achieved 100% confluence (9.1±0.2×10⁴ cells/cm²; 4.7±0.2×10⁵ cells/mL), 50% cell density occurred on day 3 (5.1±0.2×10⁴ cells/cm²; 2.6±0.2×10⁵ cells/mL) and 80% cell confluence for monolayer (3.43±0.2×10⁴ cells/cm²) was observed at day 4.

Furthermore, heMSCs harvested from 50% confluence LPCL and 100% confluence LPCL culture displayed high (80%-90%) levels of MSC makers CD73, CD90, and CD105, with low levels of CD34 and CD45, as shown in FIG. 10B.

Impact of Cell Confluence on Cytokine Secretion

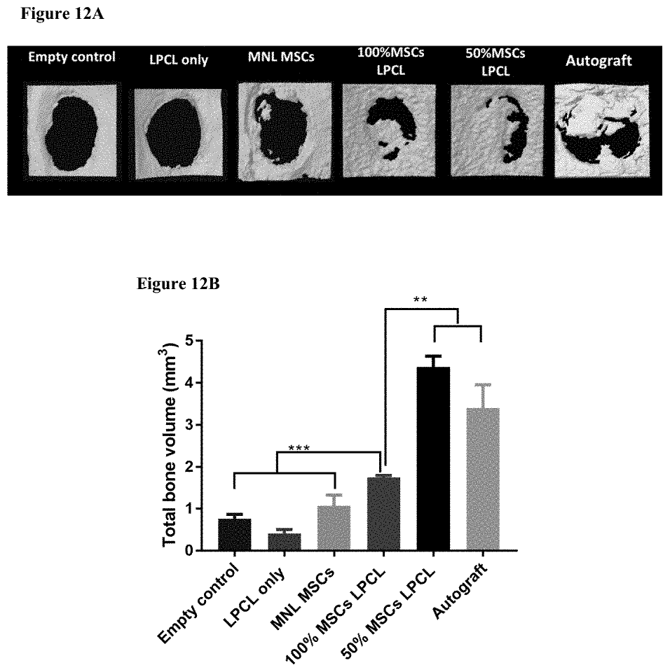

Results indicate production of IL6, IL8, SDF-1α, MCP-1, GRO-α, and VEGF-α, (among 45 cytokines tested) over the 6-days' LPCL cultures. Subconfluent (50%), mid logarithmic and confluent (100%), stationary, heMSC-covered LPCL exhibit different levels of cytokines production. Increasing cell density and the attainment of confluency, in the stationary phase, gives rise to a marked decreased in the specific production rate (lower than 1 pg/×10⁵/day) of cytokines. In particular, IL6 was significantly higher in 50% than in 100% confluent heMSCs (714.2±40.7 vs 1.7±0.2 pg/×10⁵/day; p<0.0001; FIG. 11). Similarly, IL8, SDF-1α, MCP-1, GRO-α, and VEGF-α were lower in the 100% than in the 50% confluent heMSCs (FIG. 11).

Current concepts on MSCs function is that in addition to differentiation into cells of the tissue in which they are transplanted, they also secrete several factors that play a role in the modulation of the microenvironment thus influencing tissue repair and regeneration. Paracrine signaling has been suggested as primary mechanism by which MSCs influence endogenous cells to proliferate, migrate, and inhibit apoptosis. In the present study, among 45 cytokines in an array, analysed from medium conditioned by MSCs on LPCL MC, the secretion of 6 of these was specifically upregulated in response to agitation. With respect to results from MNL control cells, agitation showed a positive effect on the secretion of IL6, IL8, VEGF, MCP-1, GRO-α and SDF-1α. All of these have been previously demonstrated to participate in bone regeneration. It was hypothesized in the present study that preconditioning of cells on microcarriers by agitation culture is an approach to increase the production of specific cytokines by cells for bone regeneration. IL6 is known for its potent roles at early stages of the bone healing process. It is a central mediator in modulating bone homeostasis. IL8 is known as an inflammatory chemokine, with potent proangiogenic properties. IL6 together with IL8 are major angiogenic factors, stimulating VEGF during fracture healing. VEGF is a paracrine factor that is most implicated in osteoblastic migration. It has been shown to be a primary regulator of both angiogenesis and vasculogenesis. Furthermore, it also regulates neutrophil release into blood circulation, during the initial stage of acute inflammation at the site of injured bone. MCP-1, a factor commonly associated with inflammatory cell-recruitment and bone remodeling, has also been known to recruit osteoclast progenitors from blood or bone marrow. GRO-1α is known as an osteoblast-derived cytokine that acts as a chemo-attractant, for growth and maintenance factors for osteoclasts, thus facilitating osteoclastogensis. SDF-1α plays an important role in endogenous stem cell migration, adhesion, homing, and recruitment from bone marrow to bone defects. It has also been shown to recruit G-protein coupled receptor CXCR-4, implying the expressing MSCs to the injury site during endochondral healing.

Ex Vivo Micro-CT Evaluation

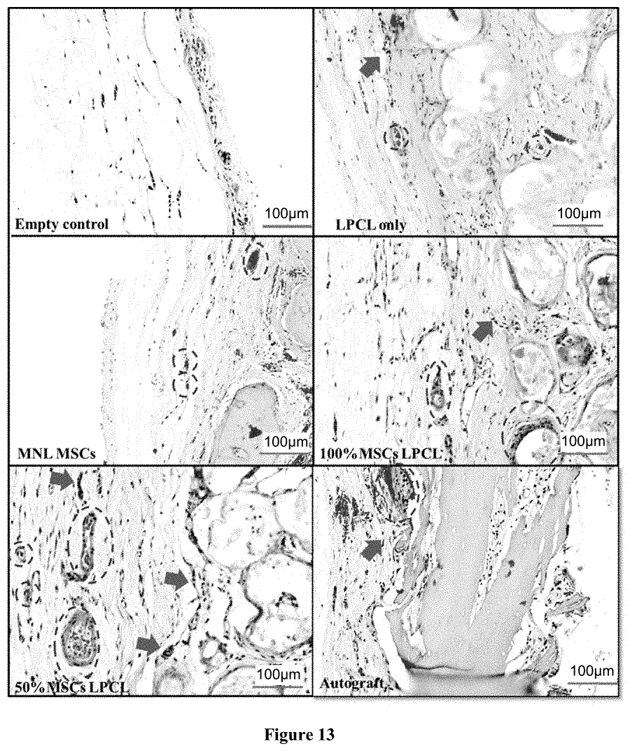

Critical-sized calvarial defects were created in rats and were utilized as a model to test the in vivo efficacy of different treatment groups: (1) empty defects as a control (Empty control), (2) defect filled with cell-free LPCL (LPCL only), (3) defect filled with MSCs harvested from MNL cultures (MNL MSCs), (4) defect filled with 50% heMSCs-covered LPCL (50% MSCs LPCL), and (5) defect filled with 100% heMSCs-covered LPCL (100% MSCs LPCL). The rats were sacrificed after 16 weeks and newly-formed bone tissue was evaluated for its volume using micro-CT scans. The induced circular bone defects and the regenerated bone tissue in the defects following diverse treatments as described above were imaged. The defect treated with cell-free LPCL yielded a low value (0.5±0.2 mm³; FIG. 12B) of bone volume. Bone regrowth in this defect is partially attributed to hydroxyapatite (HA) powder, incorporated into the implant. The MNL MSCs group gave rise to modest organized mineralized regions, which occurred at the defect's periphery, with no significant differences in overall regrown bone volume, as compared with the untreated empty defect group (p=0.09).

In contrast, the 100% MSCs LPCL group demonstrated significant mineralized tissue formation (2.1±1.3 mm³; FIG. 12B) within the defect area. This is more than two-fold higher than the MNL MSCs group (1.3±0.7 mm³; FIG. 12B). Regrown (new) bone tissue appeared to be synthesized towards the center of the defect (FIG. 12A). 50% MSCs LPCL group demonstrated dramatically better mineralized tissue formation (5.1±1.6 mm³) within the defect area, when compared to the 100% MSCs LPCL group (p<0.01). This result is comparable to the current therapeutic Gold Standard, namely Autograft of crushed bone from the original animal.

Haematoxylin and Eosin (H&E) Study

The pattern of distribution of mineralization across the defect regions as a result of diverse treatments observed in micro-CT images is consistent with histological examination using H & E stains. This further confirms the differences in bone growth volume and its distribution.

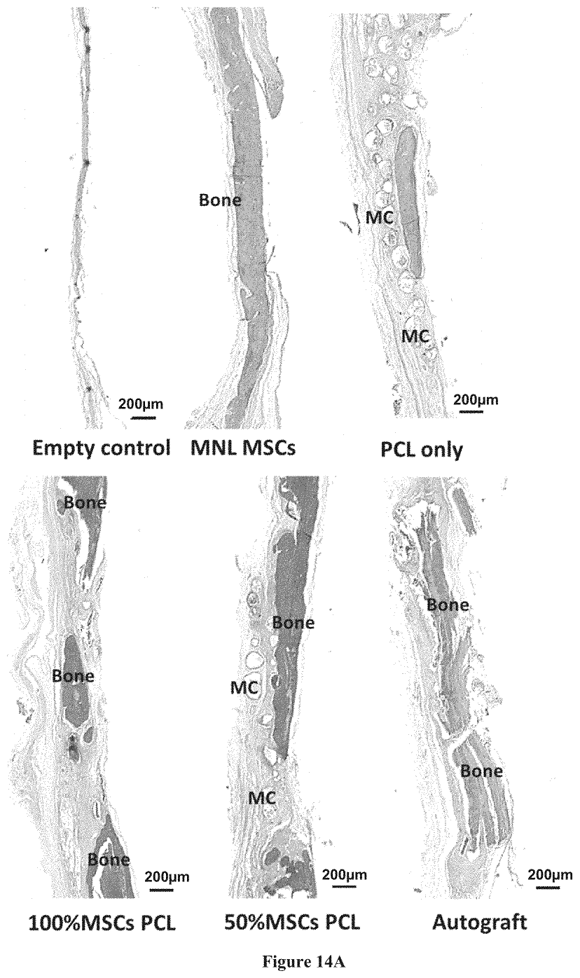

The untreated open defect showed that the original empty region, created between the old bone edges, remained unfilled. In contrast, both heMSCs-covered LPCL groups demonstrated more bone formation at the defect peripheries (FIG. 13).

In group 1, implants with fibrin gel and HA only gave rise to a loosely dispersed tissue morphology. In LPCL only group, fibrous tissue appeared between microcarriers and few microcapillaries were observed interspaced between the microcarriers. Therefore, this LPCL performed a similar function to porous scaffolds in vivo while being of a simpler design than the scaffolds.

Comparison across groups reveals that (regrown bone) tissue formation around the heMSC-covered LPCL is denser and better organized, as compared to those of MNL MSCs group. Bone formation is enhanced at the edges of the defects, gradually protruding towards the defect centre. Cells attached to the LPCL appear more flattened and elongated (indicated by an arrow), suggesting that these cells may be osteoblasts. This implies that bone remodeling occurs within the group containing heMSCs-covered LPCL. It appears that more microcapillaries occur, interspaced between the LPCL, as compared with LPCL only group.

Defects implanted with heMSCs-covered LPCL exhibited significantly more bone formation for 50% MSCs LPCL, as compared with 100% MSCs LPCL. The 50% MSCs LPCL group exhibited tissue formation where surrounding tissue was tightly associated with LPCL, thus indicating improved osteogenesis and fusion, between the LPCL and the surrounding cells. More flattened, elongated were seen (indicated by arrows), suggesting more differentiated osteoblasts in the 50% MSCs LPCL group. Moreover, more microcapillaries appeared, suggesting a higher vascularization of regenerated bone in groups containing 50% MSCs LPCL, as compared with the 100% MSCs LPCL groups.

Moreover, less microcarriers and microcarrier residue were observed to be present, in 50% MSCs LPCL as compared to LPCL only (˜78% reduction; n=6). This suggests that heMSCs enhanced the LPCL degradation process.

Masson's Trichrome Study

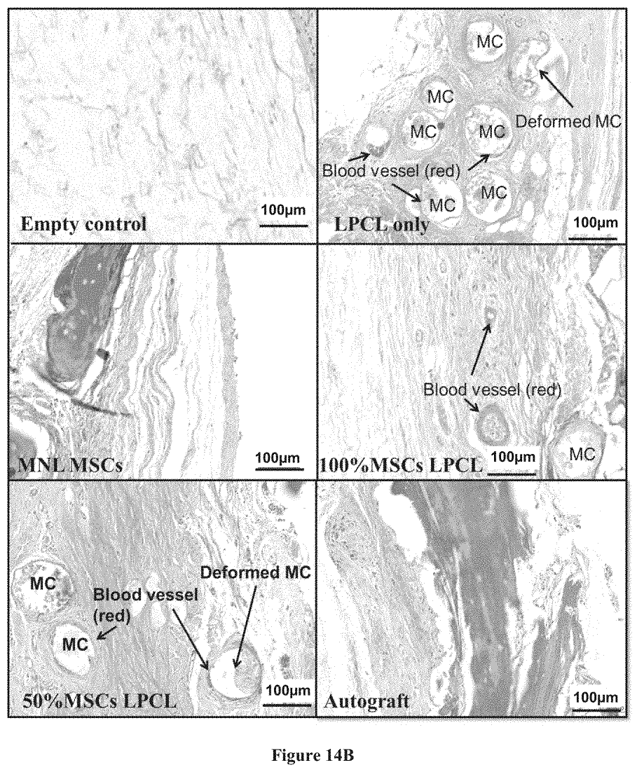

Masson's trichrome staining were performed, to differentiate between the types of tissue formed (FIG. 14). Comparing tissue formation across the groups, it was evident that tissue formation was different across the groups that introduced MSCs. In addition to the denser tissue formation, heMSCs-covered LPCL exhibited greater production of connective tissue. The observed staining is primarily associated with collagen I fibres, which are regarded as the main organic constituent of bone. More connective tissue was observed in the 50% MSCs LPCL group, than the 100% MSCs LPCL group.

### Discussion

Using Microcarriers/Bioreactor Systems

Although MSCs show promise for multiple therapeutic applications, translating these therapies is hindered by challenges in scalable and reproducible manufacturing of MSCs, at volumes that can meet clinical demand, as well as the lack of integrative bioprocesses for the expansion and delivery of MSCs. Scalable and efficient ex-vivo expansion is an important challenge, given that clinical applications require sizeable MSC doses (for example, 3-6×10⁹ cells are required for osteogenesis imperfecta treatments).

Classical methods of expanding MSCs for industrial applications in 2D monolayer flasks (usually cell stacks) offer modest cell productivity. They are less suited to culture monitoring and require laborious, time-consuming handling. In contrast, microcarriers provide a high surface-to-volume ratio, for adherent cell attachment. These supports are suitable for cell culture in controlled stirred bioreactors. Therefore, microcarrier/bioreactor systems for MSC expansion provide the advantages of scalability, automation, and improved monitoring. The present disclosure highlights a further advantage, in delivering expanded hMSC on their culture supports. Such cell/microcarrier constructs engender enhanced therapeutic efficacy, in tissue regeneration, as well as potentially for other healing applications.

Using Biodegradable/Bioimplantable Microcarriers

A common approach for bone tissue engineering is to seed MSCs on a scaffold that serves as a substrate for the cells to adhere to and as a temporary matrix inserted into the defect site to stimulate tissue regeneration. However, this approach is time-consuming because it involves three steps: cell expansion in a culture unit, followed by cell harvesting and subsequent seeding of these cells onto a second unit (the scaffold). Traditional enzymatic dissociation methods using proteases are the most common means for cell harvesting. However, they only yield 60%-70% cell recovery, depending on the MC properties. The enzymatic process may also cause reduced cell viability and high apoptotic activity, which is expected to limit the therapeutic efficacy of transplantated cells. Moreover, uniform cell seeding onto the MCs or scaffolds to attain their functional properties as tissue-engineered implants can be problematic.

The present disclosure describes the use of the biodegradable PCL MC, for chondrogentic differentiation, cartilage formation, osteogenic differentiation and bone formation. The use of a biodegradable, bioresorbable, polymer that is FDA approved for implants allows cells cultured on the microcarriers to be implanted, as cell/microcarrier constructs, in-vivo. These serve as an integral part of the tissue engineering process, in the context of bone and cartilage regeneration. This innovation may serve other tissue engineering applications, and other forms of healing (e.g. reducing inflammation) may be viable and suitable for either the microcarriers or the hMSC cell/microcarrier constructs. The benefit of not dissociating the cells from the support on which they are cultured improves viability and potentially allows more rapid adaptation to their role in the tissue engineering applications.

PCL microcarriers also offer an advantage, whereby cell harvesting and the use of scaffolds to transfer cells are not required. In this manner, high cell viability and potency can be maintained.

1. A method of manufacturing an implantable construct comprising chondrogenically differentiated cells and one or more polycaprolactone (PCL) microcarriers, the method comprising:
a) culturing mesenchymal stromal cells with one or more PCL microcarriers in a suspension culture in a mesenchymal stromal cells growth medium to allow the mesenchymal stromal cells to attach to the PCL microcarriers to form one or more mesenchymal stromal cells-PCL microcarrier complexes, wherein the suspension culture is agitated;
b) harvesting the one or more mesenchymal stromal cells-PCL microcarrier complexes from the suspension culture in a) while the suspension culture is agitated;
c) culturing the one or more mesenchymal stromal cells-PCL microcarrier complexes from b) under agitation-free and centrifugation-free conditions in the mesenchymal stromal cells growth medium;
d) culturing the one or more mesenchymal stromal cells-PCL microcarrier complexes from c) under agitation-free and centrifugation-free conditions in a chondrogenic differentiation medium to enact differentiation of the mesenchymal stromal cells into chondrogenically differentiated cells.

2. The method of claim 1, wherein the number of mesenchymal stromal cells to be cultured in a) is about 3×10⁴ to about 7×10⁴, or about 4.5×10⁴ to about 5.5×10⁴, or about 5×10⁴ per construct of PCL microcarriers.

3. The method of claim 1, wherein b) is carried out during the early log phase of a).

4. The method of claim 3, wherein the early log phase of a) is about 2.5 to about 3.5 days, or about 3 days from starting the culturing in a).

5. The method of claim 3, wherein a confluency of mesenchymal stromal cells on the microcarriers at the early log phase is at about 20% to 30%, or at about 21%.

6. The method of claim 1, wherein c) and/or d) comprises culturing the one or more mesenchymal stromal cell-microcarrier complexes in an adherent culture on a support surface.

7. The method of claim 6, wherein the support surface is a low adhesion support surface.

8. The method of claim 1, wherein c) comprises culturing the one or more mesenchymal stromal cell-microcarrier complexes for about 1 day, or about 18 to 24 hours.

9. The method of claim 1, wherein d) comprises culturing the one or more mesenchymal stromal cell-microcarrier complexes from c) for about 14 days to about 28 days, or for about 21 days to about 28 days, or for about 28 days.

10. The method of claim 1, comprising:
a) culturing about 4.5×10⁴ to about 5.5×10⁴ mesenchymal stromal cells with one construct of PCL microcarriers in a suspension culture in a mesenchymal stromal cells growth medium for about 2.5 days to about 3.5 days or until a confluency of the mesenchymal stromal cells is about 20% to about 30%, to allow the mesenchymal stromal cells to attach to the PCL microcarriers to form mesenchymal stromal cells-PCL microcarrier complexes, wherein the suspension culture is agitated;
b) harvesting the mesenchymal stromal cells-PCL microcarrier complexes from the suspension culture in a) while the suspension culture is agitated;
c) culturing the mesenchymal stromal cells-PCL microcarrier complexes from b) under agitation-free and centrifugation-free conditions in the mesenchymal stromal cells growth medium for about 0.5 day to about 1.5 days; and
d) culturing the mesenchymal stromal cells-PCL microcarrier complexes from c) under agitation-free and centrifugation-free conditions in a chondrogenic differentiation medium for about 14 days to about 28 days to enact differentiation of the mesenchymal stromal cells into chondrogenically differentiated cells.

11. An implantable construct comprising chondrogenically differentiated cells and one or more PCL microcarriers, produced using the method of claim 1.

12. (canceled)

13. The implantable construct of claim 11, wherein the DNA content per construct is about 0.5 μg to about 1.0 μg.

14. The implantable construct of claim 11, wherein the Glycosaminoglycan (GAG) content per construct is about 15 μg to about 50 μg.

15. The implantable construct of claim 11, wherein the collagen II content per construct is about 150 ng to about 500 ng.

16. The implantable construct of claim 11, wherein a GAG/DNA ratio is about 25 to about 50.

17. The implantable construct of claim 11, wherein a collagen II/DNA ratio is about 200 to about 500.

18. (canceled)

19. A method of promoting cartilage tissue regeneration in a patient in need thereof, the method comprising administering the implantable construct of claim 11 in the patient.

20. The method of claim 19, wherein administering the implantable construct comprises administering about 140 to about 150 microcarriers per mm³ of cartilage defect.

21. The method of claim 19, wherein administering the implantable construct comprises occupying about 10% to about 28% of the cartilage defect.

22. The method of claim 19, wherein administering the implantable construct comprises administering about 2000 to about 6000 cells per mm³ of cartilage defect.

23.-24. (canceled)
